SEQUENCE LISTING (4 sequences)

SEQ ID NO: 1 - DNA, 1431 bp, Homo sapiens
atgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgt

SEQ ID NO: 2 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 3 - DNA, 1440 bp, Mus sp.
atgaaaatgaagggtcttccggtcctgctgtggctgtgtgtggttgtgtgctcatcctacccattgcatgacagtgcaagggatgatgatgctggtatggagcttctgcagaaatacctagaaaactactatggccttgcaaaagatgtgaagcaatttattaagaaaaaggacagtagtcttattgtcaaaaaaattcaagaaatgcagaagttcctcgggttggagatgacagggaagctggactccaacactatggagctgatgcataagcccaggtgtggtgttcctgatgttggtggcttcagtaccttcccaggttcgccaaaatggaggaaatcccacatcacctacaggattgtgaattatacaccggatttgccaagacagagtgtggattctgccattgaaaaagctttgaaggtctgggaggaggtgaccccactcactttctccaggatctctgaaggagaggctgacataatgatctcctttgcagttggagaacatggagactttgtcccttttgatgggcctggaacagtcttggctcatgcctatgcacctggaccagggattaatggagatgctcactttgacgatgatgaacgatggacagaggatgtcactggtaccaacctattcctggttgctgctcatgaacttggccactccctgggactctaccactcagccaaggctgaagctctgatgtacccagtctacaagtcctccacagacttgtcccgtttccatctctctcaagatgatgtagatggtattcagtccctctatggaactcccacagcatcccctgatgtcctcgtggtacccaccaagtctaactctctggaacctgagacatcaccaatgtgcagctctactttgttctttgatgcagtcagcaccctccggggagaagtcctgttttttaaagacaggcacttttggcgcaaatctctcaggactcctgagcctgaattttatttgatctcttcattttggccatctcttccatccaacatggatgctgcatatgaggttactaacagagacactgttttcatttttaaaggaaatcagttctgggctatacgagggcacgaggagctagcaggttatcctaaaagcattcacaccctgggtctccctgcaaccgtgaagaagatcgatgctgccatttctaataaagagaaaaggaagacctacttctttgtagaggacaaatactggaggtttgatgagaagaaacaatccatggagccaggatttcccaggaagatagctgaggactttccaggtgttgactcaagggtggatgctgtctttgaagcatttgggtttctctacttcttcagtggatcttcgcagttggaatttgacccaaatgccaaaaaagtgacccacatattgaagagcaatagctggtttaattgttaa

SEQ ID NO: 4 - protein, 479 aa, Mus sp.
MKMKGLPVLLWLCVVVCSSYPLHDSARDDDAGMELLQKYLENYYGLAKDVKQFIKKKDSSLIVKKIQEMQKFLGLEMTGKLDSNTMELMHKPRCGVPDVGGFSTFPGSPKWRKSHITYRIVNYTPDLPRQSVDSAIEKALKVWEEVTPLTFSRISEGEADIMISFAVGEHGDFVPFDGPGTVLAHAYAPGPGINGDAHFDDDERWTEDVTGTNLFLVAAHELGHSLGLYHSAKAEALMYPVYKSSTDLSRFHLSQDDVDGIQSLYGTPTASPDVLVVPTKSNSLEPETSPMCSSTLFFDAVSTLRGEVLFFKDRHFWRKSLRTPEPEFYLISSFWPSLPSNMDAAYEVTNRDTVFIFKGNQFWAIRGHEELAGYPKSIHTLGLPATVKKIDAAISNKEKRKTYFFVEDKYWRFDEKKQSMEPGFPRKIAEDFPGVDSRVDAVFEAFGFLYFFSGSSQLEFDPNAKKVTHILKSNSWFNC

(19) United States
(12) Patent Application Publication
(10) Pub. No.: US 2019/0358305 A1
(43) Pub. Date: Nov. 28, 2019

(54) AAV-BASED GENE THERAPY FOR GLAUCOMA

(71) Applicant: The Provost, Fellows, Scholars and other Members of Board of Trinity College Dublin, Dublin (IE)

(72) Inventors: Matthew Campbell, Dublin (IE); Peter Humphries, Dublin (IE); Jeffrey O'Callaghan, Dublin (IE)

(73) Assignee: The Provost, Fellows, Scholars and other Members of Board of Trinity College Dublin, Dublin (IE)

(21) Appl. No.: 16/264,095

(22) Filed: Jan. 31, 2019

Related U.S. Application Data
(60) Provisional application No. 62/624,460, filed on Jan. 31, 2018.

(51) Int. Cl. A61K 38/48; A61K 48/00; A61P 27/06; A61K 9/00; C12N 15/86

(52) U.S. Cl. CPC A61K 38/4886; A61K 48/0083; A61P 27/06; C12Y 304/24017; C12N 15/86; A61K 48/0025; A61K 9/0048

(57) ABSTRACT

The disclosure provides compositions and methods useful for treating glaucoma. In particular, the invention provides an adeno-associated viral (AAV)-mediated gene therapy for glaucoma in which transduced cells of the eye secrete a therapeutic protein (for example, a matrix metalloproteinase) resulting in remodeling of the extracellular matrix of the trabecular meshwork of said eye.

### CROSS-REFERENCE TO RELATED APPLICATION

This application claims the benefit of U.S. Provisional Application No. 62/624,460, filed Jan. 31, 2018, the disclosure of which is hereby incorporated by reference in its entirety.

### FIELD OF THE INVENTION

The present disclosure relates generally to gene therapy for glaucoma. In particular, the disclosure relates to an adeno-associated viral (AAV)-mediated gene therapy for glaucoma in which transduced cells of the eye secrete a therapeutic protein (for example a matrix metalloproteinase).

### STATEMENT REGARDING SEQUENCE LISTING

The sequence listing associated with this application is provided in text format in lieu of a paper copy and is hereby incorporated by reference into the specification. The name of the text file containing the sequence listing is 68296_Seq_Final_2019-01-31.txt. The text file is 12.1 KB; was created on Jan. 31, 2019 and is being submitted via EFS-Web with the filing of the specification.

### BACKGROUND OF THE INVENTION

The U.S. spends $1.9 billion per annum to treat glaucoma, principally topical pressure reducing medications. Such medications often do not reduce intraocular pressure to the desired target pressure and may induce side effects in certain patients. Such patients may then undergo surgical interventions, which have associated risks and complications. Open-angle Glaucoma (OAG) and Primary Open-angle Glaucoma (POAG). See, for example, Grant, W. M., “Clinical Measurements of Aqueous Outflow,” Am. J. Ophthalmol., 1951, 34:1603-1605.

The greatest risk factor in Open-angle Glaucoma is elevated intraocular pressure, which impacts on the viability of retinal ganglion cells and tissues of the optic nerve head. Up to 6% of cases of Open-angle Glaucoma (up to 300,000 cases in the US and Europe combined) are bilaterally sub-optimally responsive to standard topically-applied pressure-reducing medications. Aqueous humor leaves the eye largely via the conventional outflow pathway—through the Trabecular Meshwork and into the Canal of Schlemm, some leaving via the uveoscleral route between the bundles of the ciliary muscles. Currently used topical formulations either decrease aqueous production by the ciliary body or enhance its movement through the uveoscleral route, none of these acting primarily on the major, conventional outflow pathway. The planned and actual use of the invention involves an adeno-associated viral (AAV)-mediated gene therapy to be deployed in those cases of treatment-resistant disease. The therapy involves injection of an AAV construct into the anterior chamber of the eye such that the virus selectively expresses a matrix metalloproteinase (for example MMP3) in the endothelial cell layer of the cornea. The enzyme is secreted into the anterior chamber of the eye and moves with the natural flow of aqueous humor through the Trabecular Meshwork™. The processed enzyme selectively degrades a series of extracellular matrix (ECM) proteins within the TM, resulting in an enhancement of movement of aqueous humor through the drainage channel. The invention represents a form of ‘molecular trabeculectomy’ and is deployable in a minimally invasive sense.

Thus, there is a long-felt yet unmet need for compositions and methods for adeno associated viral (AAV)-mediated gene therapy for glaucoma in which transduced cells of the eye secrete a therapeutic protein (for example a matrix metalloproteinase). The disclosure provides such novel compositions and methods to address and solve this need.

### SUMMARY OF THE INVENTION

The disclosure provides compositions and methods useful for treating glaucoma. In particular, the invention provides an adeno-associated viral (AAV)-mediated gene therapy for glaucoma in which transduced cells of the eye secrete a therapeutic protein (for example a matrix metalloproteinase) resulting in remodeling of the extracellular matrix of the trabecular meshwork of said eye.

In some embodiments of the compositions of the disclosure, a recombinant AAV (rAAV) vector comprises a polynucleotide sequence encoding matrix metalloproteinase 3 (MMP-3). In some embodiments, the rAAV vector comprises a single stranded genome. In some embodiments, the rAAV comprises a self-complementary genome. In some embodiments, the polynucleotide sequence encoding matrix metalloproteinase 3 (MMP-3) is operatively linked to an inducible promoter. In some embodiments, the inducible promoter is inducible by tetracycline. In some embodiments, the polynucleotide sequence encoding matrix metalloproteinase 3 (MMP-3) is operably linked to a CMV promoter.

In some embodiments of the compositions of the disclosure, the polynucleotide sequence encoding MMP-3 comprises a nucleotide sequence at least 95% identical to SEQ ID NO: 1 (human MMP-3). In an embodiment, the polynucleotide sequence encoding MMP-3 comprises a nucleotide sequence at least 95% identical to SEQ ID NO: 3 (mouse MMP-3). In some embodiments, the polynucleotide sequence encoding MMP-3 comprises the nucleotide sequence set forth in SEQ ID NO: 1. In some embodiments, the polynucleotide sequence encoding MMP-3 comprises the nucleotide sequence set forth in SEQ ID NO: 3.

In some embodiments of the compositions of the disclosure, the rAAV vector comprises the capsid from AAV1, AAV2, AAV4, AAV5, AAV6, AAV7, AAV8, AAV9, AAV10, AAV11, AAV12, AAV13, or Anc80L65. In some embodiments, the rAAV vector is comprises the capsid from AAV9. In some embodiments, the rAAV vector comprises the capsid from Anc80L65.

In some embodiments, said rAAV vector comprises a single stranded genome. In some embodiments, said rAAV vector comprises a double-stranded or self-complementary genome. In some embodiments, the rAAV vector comprises an AAV2 genome, such that the rAAV vector is an AAV-2/1, AAV-2/9 vector, AAV-2/4, AAV-2/5, AAV-2/6, AAV-2/7, AAV-2/8, AAV-2/9, AAV2/10, AAV-2/11, AAV-2/12, AAV-2/13, or AAV-2/Anc80L65. In some embodiments, the rAAV vector comprises the capsid from AAV9 and comprises the nucleotide sequence set forth in SEQ ID NO: 1 (human MMP-3). In some embodiments, the rAAV vector is of the serotype AAV9, comprises an AAV2 genome, and comprises the nucleotide sequence set forth in SEQ ID NO: 1 (human MMP-3).

In some embodiments of the compositions of the disclosure, contacting the rAAV vector to a human trabecular meshwork (HTM) monolayer increases the rate of tracer molecule flux through said monolayer by more than about 10% over the tracer molecule flux through a HTM monolayer not contacted with said rAAV.

In some embodiments of the compositions of the disclosure, contacting said rAAV vector to a human trabecular meshwork (HTM) monolayer decreases the transendothelial electrical resistance (TEER) of said monolayers by more than about 10 Ohm per cm², more than about 15 Ohm per cm², or more than about 20 Ohm per cm² over the TEER of a monolayer not contacted with said rAAV.

The disclosure provides a method of treating glaucoma in a subject suffering from glaucoma, comprising administering to an eye of the subject a therapeutically effective amount of a recombinant AAV (rAAV) comprising a polynucleotide sequence encoding matrix metalloproteinase 3 (MMP-3).

In some embodiments of the methods of the disclosure, the polynucleotide sequence encoding MMP-3 comprises a nucleotide sequence at least 95% identical to SEQ ID NO: 1. In some embodiments of the methods of the disclosure, the rAAV vector is of the serotype AAV9. In some embodiments of the methods of the disclosure, the rAAV comprises the nucleotide sequence set forth in SEQ ID NO: 1.

In some embodiments of the methods of the disclosure, administering the rAAV to said eye increases permeability of the extracellular matrix of the trabecular meshwork of said eye. In some embodiments, administering the rAAV to said eye decreases outflow resistance of said eye. In some embodiments, administering the rAAV to said eye increases outflow of said eye. In some embodiments, administering the rAAV to said eye decreases intraocular pressure (IOP) of said eye. In some embodiments, the rAAV is administered by intracameral, intravitreal, subretinal, or suprachoroidal inoculation. In some embodiments, the rAAV is administered by canaloplasty. In some embodiments, the rAAV is administered within an hour prior to or following cataract removal or intraocular lens placement.

The disclosure further provides a method of lowering ocular pressure in a subject in need thereof, comprising administering to said eye a protein capable of remodeling or degrading the extracellular matrix, or a polynucleotide sequence encoding the protein. In some embodiments of the methods of the disclosure, the protein is a matrix metalloproteinase. In some embodiments of the methods of the disclosure, administering the protein or the polynucleotide to said eye increases permeability of the extracellular matrix of the trabecular meshwork of said eye.

The disclosure further provides a method of treating a vision disorder in a mammal, comprising injecting a therapeutic composition comprising an rAAV vector into the anterior chamber of said mammal's eye, wherein the rAAV vector transduces cells nearby or in contact with the anterior chamber; wherein the transduced cells secrete a therapeutic protein; wherein the therapeutic protein modifies the extracellular matrix of the trabecular meshwork of said mammal's eye; and wherein said method improves a symptom, biomarker, or treats said vision disorder in said mammal.

In some embodiments of the methods of the disclosure, said rAAV is of the serotype AAV1, AAV2, AAV4, AAV5, AAV6, AAV7, AAV8, AAV9, AAV10, AAV11, AAV12, AAV13, or Anc80L65. In some embodiments of the methods of the disclosure, said therapeutic protein is a matrix metalloproteinase (MMP). In some embodiments, said MMP is a mammalian MMP-3, such as murine MMP-3 or human MMP-3.

In some embodiments of the methods of the disclosure, the intraocular pressure (IOP) of said mammal's eye is decreased by more than 1, 2, 3, 4, or 5 mmHg. In some embodiments, the outflow rate of said mammal's eye is increased by more than 1, 2, 3, 4, 5, 10, or 15 nl/min/mmHg. In some embodiments, optically empty length in the trabecular meshwork of said mammal's eye is increased by more than about 5, 10, 15, 20, 25, 30, 35, 40, 45, or 50%. In some embodiments, the therapeutic composition comprises more than about 1E8, 1E9, 1E10, 1E11, 1E12 genomes of the rAAV vector per dose (i.e., volume of therapeutic composition injected). In some embodiments, the therapeutic composition comprises concentrations of more than approximately 1E10, 1E11, 1E12, or 1E13 genomes of the rAAV vector per mL. In some embodiments of the methods of the disclosure, the transduced cells are cells of the corneal endothelium. In some embodiments, MMP-3 concentration in aqueous humor of said eye is increased by about 0.49 ng/ml or greater. In some embodiments, MMP-3 activity in aqueous humor of said eye is increased by about 5.34 mU or greater. In some embodiments, the corneal thickness of said mammal is unchanged following treatment.

The disclosure further provides a method of lowering intraocular pressure in a mammal comprising administering to an eye of the mammal a therapeutically effective amount of a recombinant AAV (rAAV) comprising a polynucleotide sequence encoding a matrix metalloproteinase (MMP). In some embodiments of the methods of the disclosure, the MMP is MMP-3.

The foregoing paragraphs are not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. The invention includes, as an additional aspect, all embodiments of the invention narrower in scope in any way than the variations defined by specific paragraphs above. For example, where certain aspects of the invention that are described as a genus, it should be understood that every member of a genus is, individually, an aspect of the invention.

### BRIEF DESCRIPTION OF THE DRAWINGS

FIGS. 1A-1F. MMP-3 concentration in glaucomatous AH and the resulting effect on SCEC and HTM monolayers. FIG. 1A. MMP-3 concentrations in the media of SCEC monolayers treated with either cataract (control) or POAG human AH showed no significant difference after 24 h. FIG. 1B. POAG aqueous-treated SC media samples from FIG. 1A were found to have an average change in MMP-3 proteolytic activity of −0.15 [−0.28, −0.02] mU/ml compared to control media. FIG. 1C. Addition of POAG aqueous humor onto SC monolayers resulted in an average increase in TEER of 102% compared to controls. FIG. 1D. Treatment of HTM cells with human aqueous also increased TEER value. FIGS. 1E-1F. SCEC and HTM subjected to AH were tested for cellular permeability using a FITC-Dextran flux assay respectively. Decreased permeability to a 70 kDa dextran was observed in response to POAG rather than cataract AH. Graphs show mean with 95% CI error bars. FIGS. 1A-1F were analysed with a Student's t-test. NS ¼ non-significant. Symbols *, ** and *** denote P values of <0.05, <0.01 and <0.001, respectively.

FIGS. 2A-2F. Effect of recombinant human MMP-3 on paracellular permeability in HTM and SCEC cell monolayers. SCEC and HTM cells were treated with 10 ng/ml recombinant MMP-3 for 24 h, using PBS and inactivated MMP-3 (incubation with TIMP-1, MMP(−)) as vehicle and negative controls respectively. SCEC (FIG. 2A) and HTM (FIG. 2B) both show reductions in TEER values after treatment of 4.6 [2.9, 6.2] and 5 [2.2, 7.8] Ohms·cm² respectively. Permeability to a 70 kDa dextran was increased in treated cells (MMP(+)) in both SCEC (FIG. 2C) and HTM (FIG. 2D). FIG. 2E. An average viability of 85% was expected for SCEC with MMP-3 concentrations up to 36 ng/ml. FIG. 2F. 85% viability is retained on average in HTM cells at concentrations up to 151 ng/ml MMP-3. FIG. 2A, FIG. 2C, and FIG. 2E represent SCEC data, whereas FIG. 2B, FIG. 2D, and FIG. 2F represent HTM data.

FIGS. 3A-3H. Remodeling of ECM components in SCEC and HTM cell monolayers. Immunocytochemistry shows various remodeling artefacts on core ECM components in SCEC and HTM cells in response to MMP-3 treatment. FIG. 3A, FIG. 3B. Collagen IV appears to have reduced intensity in both cell types after treatment. Collagen IV is concentrated around cells in controls but shows reduced spread after treatment, fibrils barely protruding past the cell nuclei. FIG. 3C. Alpha smooth muscle fibers extend the width of the cell towards a neighboring cell. Treated samples show that these fiber bundles have constricted, leading to multiple thin connections between cells. FIG. 3D. HTM F-actin staining depicts a slight thinning of filament bundles and a reduction of filament branching post MMP-3 treatment. FIG. 3E, FIG. 3F. Laminin expression exhibits a modest reduction in staining intensity in both cell types, and a reduction in network complexity in TM cells. FIG. 3G, FIG. 3H. Fibronectin was visualized after decellularization, depicting linear and organized strands in PBS controls, as denoted by an asterisk. Treatment groups lacked a linear network, and instead showed a disjointed, porous network. Scale bars represent 50 μm. FIG. 3A, FIG. 3C, FIG. 2E, FIG. 3G present results with SCEC. FIG. 3B, FIG. 3D, FIG. 2F, and FIG. 3H present results with HTM.

FIGS. 4A-4E. AAV-2/9 mediated MMP-3 expression in the corneal endothelium. FIG. 4A. Diagrams illustrating the therapeutic concept addressed in this study. AAV-2/9 transduces the corneal endothelium upon intracameral inoculation (left). MMP-3 molecules are secreted into the AH from this location and are transported toward the outflow tissue by the natural flow of the aqueous (right). FIG. 4B. A schematic diagram of the AAV-2/9 vector used for the expression of either eGFP or MMP-3. Murine MMP-3 cDNA was sub-cloned into the pAAV-MCS plasmid and constitutively driven by a CMV promoter (AAV-MMP-3). FIG. 4C. Immunohistochemistry images of corneas from WT murine eyes intracamerally inoculated with AAV-2/9 expressing eGFP. AAV virus containing a CMV promoter demonstrates transduction and expression at the corneal endothelium (marked with arrows). Using the AAV-MMP-3 virus, MMP-3 was detected at the corneal endothelium in treated eyes only, denoted by arrows. FIG. 4D. ELISA was performed on murine AH 4 weeks post-injection of virus. MMP-3 concentrations had increased by an average of 0.49 [0.11, 0.87] ng/ml in AAV-MMP-3 treated eyes (paired Student's t-test). FIG. 4E. Aqueous MMP-3 activity was significantly increased by an average of 5.34 [1.12, 9.57] mU in AAV-MMP-3 treated eyes. Scale bars represent 50 μm. Asterisk symbol denotes a P value of <0.05.

FIGS. 5A-5E. Effect of ECM remodelling on outflow facility and IOP. FIG. 5A. ‘Cello’ plot depicting individual outflow facility values for eyes at 8 mmHg (Cr) and statistical distribution of both control (AAV-Null) and experimental (AAV-MMP-3) groups. Each point represents a single eye with 95% CI on Cr. Log normal distribution is shown, with the central white band showing the geometric mean and the thinner white bands showing two geometric standard deviations from the mean. The shaded region represents the 95% CI on the mean. FIG. 5B. Paired outflow facility plot. Each inner point represents an eye pair, with log-transformed facilities of the control eye plotted on the x axis, and treated eye on the y axis. Outer dark gray and light gray ellipses show uncertainties generated from fitting the data to a model, intra-individual and cannulation variability respectively. Average increase is denoted by the gray line, enclosed by a grey 95% CI, indicating significantly increased facility (does not overlap the gray unity line). FIG. 5C. Box plots showing the change in IOP in treated and control eyes. Boxes show interquartile range and error bars represent the 5th and 95th percentiles. A significant reduction in IOP is observed in AAV-MMP-3 treated eyes (Wilcoxon signed-rank test). FIGS. 5D-5E. Cello (FIG. 5D) and paired facility (FIG. 5E) plots for inducible AAV data sets.

FIGS. 6A-6G. Transmission electron microscopy (TEM) analysis of ECM remodeling in outflow tissues. Semi-thin sections of the iridocorneal angle in mouse eyes treated with either AAV-Null (FIG. 6A) or AAV-MMP-3 (FIG. 6B). AAV-MMP-3 treated eyes show greater inter-trabecular spaces in outer trabecular meshwork (TM) than controls. Scale bar denotes 50 μm. FIGS. 6C-6D. Transmission electron micrograph of the inner wall of Schlemm's Canal (SC) and the outer TM. FIG. 6C. Control eye illustrating normal attachment between foot-like extensions of the inner wall endothelium and sub-endothelial cells (arrowheads), as well as with the discontinuous basement-membrane material underlying the inner wall endothelium (arrows). FIG. 6D. Representative TEM image of an MMP-3 treated eye showing a disconnection of the inner wall endothelium from the sub-endothelial cells and the ECM (arrowheads). The widened sub-endothelial region lacks basement-membrane material and other ECM components. FIGS. 6E-6F. Higher magnification of the inner wall of a treated eye. FIG. 6E. Foot-like extensions of the inner wall endothelium have disconnected from the sub-endothelial cells and the ECM (arrowheads), and the lack of ECM in this region is shown. FIG. 6F. In other regions of treated eyes, clumps of presumably degraded ECM-material are localized underneath the inner wall of SC (asterisk). Such clumps of ECM are not present in controls. Scale bars are denoted on each image. FIG. 6G. Morphometric measurements of the optically empty space immediately underlying SC from four regions of contralateral eyes treated with AAV-MMP-3 (gray data points) or AAV-Null (darker gray data points). Bars indicate average values for each eye. Contralateral eyes are presented immediately next to one another.

FIGS. 7A-7F. IOP response to dexamethasone and MMP-3. IOP over the experimental timecourse in Dex-treated animals (FIG. 7A) versus controls (FIG. 7B). Gray line represents the mean trend in IOP of iGFP treated eye and the darker gray line represents the contralateral iMMP-3 treatment. Error bars are determined by 95% CI. Total change in IOP between initial and final timepoints is represented for dex-treated (FIG. 7C) and control animals (FIG. 7D). Eyes were compared to a median basal change of 0 and to its contralateral counterpart. Median IOP of the final timepoint was assessed between contralateral eyes of each group (FIGS. 7E-7F). Comparisons were made using a Wilcoxon signed rank matched pairs test. IOP was significantly reduced in response to MMP-3 treatment in dexamethasone treated animals only (FIG. 7E) as compared with control animals (FIG. 7F).

FIGS. 8A-8B. Outflow facility in response to dexamethasone and MMP-3. Cello plots depicting paired analysis between iMMP-3 and iGFP treated eyes in both the dex treated cohort (FIG. 8A) and the cyclodextrin control group (FIG. 8B). Average percentage facility difference is denoted by the white line, with the dark shading as the 95% CI of the mean. Individual data points are plotted along with their own 95% CIs. In the dex induced model (FIG. 8A), MMP-3 treatment increases outflow facility by 28%, and by 20% in the control cohort (FIG. 8B).

FIGS. 9A-9D. Quantification of ECM remodelling and degradation. Western blot analysis was performed on PBS and MMP-3 treated samples of (FIG. 9A) SC cells, (FIG. 9B) SC media, (FIG. 9C) HTM cells, and (FIG. 9D) HTM media. Significant degradation of collagen IV, α-SMA and laminin is apparent in cell lysates only. No α-SMA was detected in media samples. ‘+’ denotes a positive control lane containing a cell lysate sample. Bars represent mean fold change with 95% confidence intervals.

FIG. 10. Morphometric analysis of the optically empty space underlying the inner wall endothelium of SC. The anterior-posterior length of the inner wall was examined in 4 regions per eye at 10,000× magnification. Optically empty spaces (light gray zones) were identified, along with extracellular matrix (ECM) where the inner wall cell contacted basement membrane material, elastic fibres or amorphous material (darker gray zones). The ratio of optically empty length to total length (optically empty+ECM length) was defined as the percentage optically open length, as shown in FIG. 6G.

### DETAILED DESCRIPTION

### 1. Introduction

The disclosure provides compositions and methods useful for treating a vision disorder, lowering ocular pressure, treating glaucoma, or treating open-angle glaucoma. In particular, the invention provides an adeno-associated viral (AAV)-mediated gene therapy for glaucoma in which transduced cells of the eye secrete a therapeutic protein (for example a matrix metalloproteinase) resulting in remodeling of the extracellular matrix of the trabecular meshwork of said eye. In most cases, the therapeutic protein will be therapeutic that is secreted by the target cell but the disclosure also envisions providing a transgene encoding an intracellular signaling molecule, an siRNA or shRNA, or other macromolecule regulator of cellular function; or alternatively providing a gene editing system such as a CRISPR system, and thereby indirectly inducing secretion of proteins to remodel the extracellular matrix of the trabecular meshwork. In particular, the methods of treatment may include administering a recombinant AAV vector that delivers to ocular cells a transgene for a therapeutic protein, such as, in a preferred embodiment, a matrix metalloproteinase including MMP-3 or another matrix metalloproteinase.

One form of glaucoma that may be treated with the disclosed rAAV vectors is Open-angle Glaucoma (OAG). The greatest risk factor in OAP is elevated intraocular pressure, which impacts on the viability of retinal ganglion cells and tissues of the optic nerve head. Up to 6% of cases of Open-angle Glaucoma (up to 300,000 cases in the US and Europe combined) are bilaterally sub-optimally responsive to standard topically-applied pressure-reducing medications. Aqueous humor (AH) leaves the eye largely via the conventional outflow pathway—through the Trabecular Meshwork and into the Canal of Schlemm, some leaving via the uveoscleral route between the bundles of the ciliary muscles. Currently used topical formulations either decrease aqueous production by the ciliary body or enhance its movement through the uveoscleral route, none of these acting primarily on the major, conventional outflow pathway. The planned and actual use of some embodiments of the present disclosure related to a recombinant adeno-associated viral (rAAV)-mediated gene therapy to be deployed in those cases of treatment-resistant disease. The therapy involves injection of a rAAV construct into the anterior chamber of the eye such that the virus selectively expresses a therapeutic protein such as an enzyme or a matrix metalloproteinase (for example MMP-3) in the endothelial cell layer of the cornea. As used herein, “therapeutic protein” may refer generally to proteins with therapeutic potential in vision conditions, or to an enzyme, or to a matrix metalloproteinase, or most specifically to MMP-3. The therapeutic protein may be secreted into the anterior chamber of the eye and move with the natural flow of aqueous humor to, and through, the Trabecular Meshwork™. The therapeutic protein may selectively modify, remodel, or degrade a series of extracellular matrix (ECM) proteins within the TM, resulting in an enhancement of movement of aqueous humor through the drainage channel. The disclosed methods represent a form of ‘molecular trabeculectomy.’ One advantage of the disclosed methods is that they are deployable in a minimally invasive sense.

In the body, matrix metalloproteinases (MMPs) contribute to conventional aqueous humor outflow homeostasis in their capacity to remodel extracellular matrices of the Trabecular Meshwork, having direct impact on aqueous outflow resistance and intraocular pressure (IOP). We have discovered that a single intracameral administration (inoculation directly into the anterior chamber of the eye) of AAV-2/9 containing a CMV-driven MMP-3 gene into mice results in efficient transduction of corneal endothelium and an increase in aqueous activity of MMP-3. AAV-mediated expression of MMP-3 increases outflow facility and decreases IOP. Controlled expression using an inducible promoter activated by topical administration of doxycycline has a similar effect. Ultrastructural analysis of MMP-3-treated matrices by transmission electron microscopy reveals remodeling and degradation of ECM components within TM juxtacanalicular tissues (JXT), these data demonstrating that AAV-mediated MMP-3 secretion from corneal endothelium has significant therapeutic potential as a gene therapy for those cases of glaucoma that are sub-optimally responsive to currently available pressure-reducing medications.

The disclosed methods solve various problems with prior methods for treating visual conditions such as glaucoma. While surgical interventions are available for those individuals sub-optimally responsive to topical pressure-reducing medications (e.g., Trabeculectomy, Trabeculoplasy, Canaloplasty, mini-shunt implantation), there are significant limitations or complications. For example, trabeculectomy and trabeculoplasty fail in up to 15% and 40% of patients, respectively, and cataract and increased IOP can occur in up to 20% of patients receiving canaloplasty. In a minimally invasive genetic approach, we have discovered that a gene therapy approach achieves a “molecular trabeculectomy,” which enhances aqueous outflow from the eye and reduces IOP.

The present disclosure provides methods for molecular biological targeting of the trabecular meshwork in visual conditions, such as glaucoma and particularly OAG. Embodiments of the present disclosure may be used minimally invasive procedures, possibly requiring a single rAAV injection into the anterior chamber of the eye. rAAV is now widely accepted as an ocular gene delivery system. The present inventors have disclosed that only intracameral inoculation (inoculation into the anterior chamber of the eye) is required, a much safer and simpler procedure compared to the sub-retinal inoculations needed in gene therapies for retinal degenerations.

### 2. References and Definitions

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world. In certain aspects, the present disclosure relates to O'Callaghan J. et al., “Therapeutic Potential of AAV-mediated MMP-3 Secretion from Corneal Endothelium in Treating Glaucoma,” Human Mol. Genet., 2017 Apr. 1; 26(7):1230-1246. Doi: 10.1093/hmg/ddx028. PMID: 28158775.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

### 2.1 Adeno-Associated Virus (AAV)

As used herein, the term “AAV” is a standard abbreviation for adeno-associated virus or a recombinant vector thereof. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including two 145 nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13(1):67-76 (2006); and the AAV-11 genome is provided in Virology, 330(2):375-383 (2004). The sequence of the AAV rh.74 genome is provided in U.S. Pat. No. 9,434,928, incorporated herein by reference. The sequence of ancenstral AAVs including AAV.Anc80, AAV.Anc80L65 and their derivatives are described in WO 2015/054653A2 and Wang et al., “Single stranded adeno-associated virus achieves efficient gene transfer to anterior segment in the mouse eye.” PLoS One. 2017 Aug. 1; 12(8):e0182473. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Curr. Top. Microbiol. Immunol. 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

### 2.3 Matrix Metalloproteinases

As used herein, the terms “matrix metalloproteinases” or “MMPs” refers zinc- and calcium-dependent enzymes that are capable of degrading the constituents of the components of the extracellular matrix such as collagens, proteoglycans, and glycoproteins. Of the many classes of MMPs, MMP-3 (stromelysin-1) presents itself as an attractive candidate for targeting the ECM of outflow tissues. MMP-3 possesses a vast proteolytic target profile including type IV collagen, fibronectin, laminin, elastin, and proteoglycans, all of which are present in the meshwork and JCT regions of the outflow tissues, making this MMP of particular interest. In addition, MMP-3 can also activate other MMPs, including MMP-1 and MMP-9, further assisting in the remodeling of ECM components. In some cases, the recombinant AAV (rAAV) vectors comprise a nucleic acid molecule encoding matrix metalloproteinase (e.g., SEQ ID NO: 1), and one or more AAV ITRs flanking the nucleic acid molecule.

TABLE 1
Non-Limiting Examples of Matrix Metalloproteinase Sequences

| | | SEQ |
|---|---|---|
| Sequence description | Sequence | ID NO |
| Human MMP-3 | ATGAAGAGTCTTCCAATCCTACTGTTGCTGTGCGTGGCAG | 1 |
| polynucleotide | TTTGCTCAGCCTATCCATTGGATGGAGCTGCAAGGGGTGA | |
| sequence, | GGACACCAGCATGAACCTTGTTCAGAAATATCTAGAAAAC | |
| GenBank | TACTACGACCTCAAAAAAGATGTGAAACAGTTTGTTAGGA | |
| NM_002422.4 | GAAAGGACAGTGGTCCTGTTGTTAAAAAAATCCGAGAAAT | |
| | GCAGAAGTTCCTTGGATTGGAGGTGACGGGGAAGCTGGAC | |
| | TCCGACACTCTGGAGGTGATGCGCAAGCCCAGGTGTGGAG | |
| | TTCCTGATGTTGGTCACTTCAGAACCTTTCCTGGCATCCC | |
| | GAAGTGGAGGAAAACCCACCTTACATACAGGATTGTGAAT | |
| | TATACACCAGATTTGCCAAAAGATGCTGTTGATTCTGCTG | |
| | TTGAGAAAGCTCTGAAAGTCTGGGAAGAGGTGACTCCACT | |
| | CACATTCTCCAGGCTGTATGAAGGAGAGGCTGATATAATG | |
| | ATCTCTTTTGCAGTTAGAGAACATGGAGACTTTTACCCTT | |
| | TTGATGGACCTGGAAATGTTTTGGCCCATGCCTATGCCCC | |
| | TGGGCCAGGGATTAATGGAGATGCCCACTTTGATGATGAT | |
| | GAACAATGGACAAAGGATACAACAGGGACCAATTTATTTC | |
| | TCGTTGCTGCTCATGAAATTGGCCACTCCCTGGGTCTCTT | |
| | TCACTCAGCCAACACTGAAGCTTTGATGTACCCACTCTAT | |
| | CACTCACTCACAGACCTGACTCGGTTCCGCCTGTCTCAAG | |
| | ATGATATAAATGGCATTCAGTCCCTCTATGGACCTCCCCC | |
| | TGACTCCCCTGAGACCCCCCTGGTACCCACGGAACCTGTC | |
| | CCTCCAGAACCTGGGACGCCAGCCAACTGTGATCCTGCTT | |
| | TGTCCTTTGATGCTGTCAGCACTCTGAGGGGAGAAATCCT | |
| | GATCTTTAAAGACAGGCACTTTTGGCGCAAATCCCTCAGG | |
| | AAGCTTGAACCTGAATTGCATTTGATCTCTTCATTTTGGC | |
| | CATCTCTTCCTTCAGGCGTGGATGCCGCATATGAAGTTAC | |
| | TAGCAAGGACCTCGTTTTCATTTTTAAAGGAAATCAATTC | |
| | TGGGCTATCAGAGGAAATGAGGTACGAGCTGGATACCCAA | |
| | GAGGCATCCACACCCTAGGTTTCCCTCCAACCGTGAGGAA | |
| | AATCGATGCAGCCATTTCTGATAAGGAAAAGAACAAAACA | |
| | TATTTCTTTGTAGAGGACAAATACTGGAGATTTGATGAGA | |
| | AGAGAAATTCCATGGAGCCAGGCTTTCCCAAGCAAATAGC | |
| | TGAAGACTTTCCAGGGATTGACTCAAAGATTGATGCTGTT | |
| | TTTGAAGAATTTGGGTTCTTTTATTTCTTTACTGGATCTT | |
| | CACAGTTGGAGTTTGACCCAAATGCAAAGAAAGTGACACA | |
| | CACTTTGAAGAGTAACAGCTGGCTTAATTGT | |
| Human MMP-3 | MKSLPILLLLCVAVCSAYPLDGAARGEDTSMNLVQKYLEN | 2 |
| amino acid | YYDLKKDVKQFVRRKDSGPVVKKIREMQKFLGLEVTGKLD | |
| sequence, | SDTLEVMRKPRCGVPDVGHFRTFPGIPKWRKTHLTYRIVN | |
| GenBank | YTPDLPKDAVDSAVEKALKVWEEVTPLTFSRLYEGEADIM | |
| NP_002413.1 | ISFAVREHGDFYPFDGPGNVLAHAYAPGPGINGDAHFDDD | |
| | EQWTKDTTGTNLFLVAAHEIGHSLGLFHSANTEALMYPLY | |
| | HSLTDLTRFRLSQDDINGIQSLYGPPPDSPETPLVPTEPV | |
| | PPEPGTPANCDPALSFDAVSTLRGEILIFKDRHFWRKSLR | |
| | KLEPELHLISSFWPSLPSGVDAAYEVTSKDLVFIFKGNQF | |
| | WAIRGNEVRAGYPRGIHTLGFPPTVRKIDAAISDKEKNKT | |
| | YFFVEDKYWRFDEKRNSMEPGFPKQIAEDFPGIDSKIDAV | |
| | FEEFGFFYFFTGSSQLEFDPNAKKVTHTLKSNSWLNC | |
| Mouse MMP-3 | ATGAAAATGAAGGGTCTTCCGGTCCTGCTGTGGCTGTGTG | 3 |
| polynucleotide | TGGTTGTGTGCTCATCCTACCCATTGCATGACAGTGCAAG | |
| sequence, | GGATGATGATGCTGGTATGGAGCTTCTGCAGAAATACCTA | |
| GenBank | GAAAACTACTATGGCCTTGCAAAAGATGTGAAGCAATTTA | |
| NM_010809.2 | TTAAGAAAAAGGACAGTAGTCTTATTGTCAAAAAAATTCA | |
| | AGAAATGCAGAAGTTCCTCGGGTTGGAGATGACAGGGAAG | |
| | CTGGACTCCAACACTATGGAGCTGATGCATAAGCCCAGGT | |
| | GTGGTGTTCCTGATGTTGGTGGCTTCAGTACCTTCCCAGG | |
| | TTCGCCAAAATGGAGGAAATCCCACATCACCTACAGGATT | |
| | GTGAATTATACACCGGATTTGCCAAGACAGAGTGTGGATT | |
| | CTGCCATTGAAAAAGCTTTGAAGGTCTGGGAGGAGGTGAC | |
| | CCCACTCACTTTCTCCAGGATCTCTGAAGGAGAGGCTGAC | |
| | ATAATGATCTCCTTTGCAGTTGGAGAACATGGAGACTTTG | |
| | TCCCTTTTGATGGGCCTGGAACAGTCTTGGCTCATGCCTA | |
| | TGCACCTGGACCAGGGATTAATGGAGATGCTCACTTTGAC | |
| | GATGATGAACGATGGACAGAGGATGTCACTGGTACCAACC | |
| | TATTCCTGGTTGCTGCTCATGAACTTGGCCACTCCCTGGG | |
| | ACTCTACCACTCAGCCAAGGCTGAAGCTCTGATGTACCCA | |
| | GTCTACAAGTCCTCCACAGACTTGTCCCGTTTCCATCTCT | |
| | CTCAAGATGATGTAGATGGTATTCAGTCCCTCTATGGAAC | |
| | TCCCACAGCATCCCCTGATGTCCTCGTGGTACCCACCAAG | |
| | TCTAACTCTCTGGAACCTGAGACATCACCAATGTGCAGCT | |
| | CTACTTTGTTCTTTGATGCAGTCAGCACCCTCCGGGGAGA | |
| | AGTCCTGTTTTTTAAAGACAGGCACTTTTGGCGCAAATCT | |
| | CTCAGGACTCCTGAGCCTGAATTTTATTTGATCTCTTCAT | |
| | TTTGGCCATCTCTTCCATCCAACATGGATGCTGCATATGA | |
| | GGTTACTAACAGAGACACTGTTTTCATTTTTAAAGGAAAT | |
| | CAGTTCTGGGCTATACGAGGGCACGAGGAGCTAGCAGGTT | |
| | ATCCTAAAAGCATTCACACCCTGGGTCTCCCTGCAACCGT | |
| | GAAGAAGATCGATGCTGCCATTTCTAATAAAGAGAAAAGG | |
| | AAGACCTACTTCTTTGTAGAGGACAAATACTGGAGGTTTG | |
| | ATGAGAAGAAACAATCCATGGAGCCAGGATTTCCCAGGAA | |
| | GATAGCTGAGGACTTTCCAGGTGTTGACTCAAGGGTGGAT | |
| | GCTGTCTTTGAAGCATTTGGGTTTCTCTACTTCTTCAGTG | |
| | GATCTTCGCAGTTGGAATTTGACCCAAATGCCAAAAAAGT | |
| | GACCCACATATTGAAGAGCAATAGCTGGTTTAATTGTTAA | |
| Mouse MMP-3 | MKMKGLPVLLWLCVVVCSSYPLHDSARDDDAGMELLQKYL | 4 |
| amino acid | ENYYGLAKDVKQFIKKKDSSLIVKKIQEMQKFLGLEMTGK | |
| sequence, | LDSNTMELMHKPRCGVPDVGGFSTFPGSPKWRKSHITYRI | |
| GenBank | VNYTPDLPRQSVDSAIEKALKVWEEVTPLTFSRISEGEAD | |
| NP_034939.1 | IMISFAVGEHGDFVPFDGPGTVLAHAYAPGPGINGDAHFD | |
| | DDERWTEDVTGTNLFLVAAHELGHSLGLYHSAKAEALMYP | |
| | VYKSSTDLSRFHLSQDDVDGIQSLYGTPTASPDVLVVPTK | |
| | SNSLEPETSPMCSSTLFFDAVSTLRGEVLFFKDRHFWRKS | |
| | LRTPEPEFYLISSFWPSLPSNMDAAYEVTNRDTVFIFKGN | |
| | QFWAIRGHEELAGYPKSIHTLGLPATVKKIDAAISNKEKR | |
| | KTYFFVEDKYWRFDEKKQSMEPGFPRKIAEDFPGVDSRVD | |
| | AVFEAFGFLYFFSGSSQLEFDPNAKKVTHILKSNSWFNC | |

### 2.4 AAV Serotypes and Genomes

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV-10, AAV-11, AAV-12, AAV-13, and Anc80L65. Production of pseudotyped rAAV is disclosed in, for example, WO 01/83692. Other types of rAAV variants, for example rAAV with capsid mutations, are also contemplated. See, for example, Marsic et al., Molecular Therapy, 22(11):1900-1909 (2014). The nucleotide sequences of the genomes of various AAV serotypes are known in the art. To promote eye-specific expression, AAV6, AAV8 or AAV9 may be used.

In some cases, the rAAV comprises a self-complementary genome. As defined herein, an rAAV comprising a “self-complementary” or “double stranded” genome refers to an rAAV which has been engineered such that the coding region of the rAAV is configure to form an intra-molecular double-stranded DNA template, as described in McCarty et al. Self-complementary recombinant adeno-associated virus (scAAV) vectors promote efficient transduction independently of DNA synthesis. Gene Ther. 8 (16):1248-1254 (2001). The present disclosure contemplates the use, in some cases, of an rAAV comprising a self-complementary genome because upon infection (such as transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (≈2.4 kb).

In other cases, the rAAV vector comprises a single stranded genome. As defined herein, a “single standard” genome refers to a genome that is not self-complementary. In most cases, non-recombinant AAVs are have singled stranded DNA genomes. There have been some indications that rAAVs should be scAAVs to achieve efficient transduction of cells, such as ocular cells. The present disclosure contemplates, however, rAAV vectors that may have singled stranded genomes, rather than self-complementary genomes, with the understanding that other genetic modifications of the rAAV vector may be beneficial to obtain optimal gene transcription in target cells. In some cases, the present disclosure relates to single-stranded rAAV vectors capable of achieving efficient gene transfer to anterior segment in the mouse eye. See, Wang et al., “Single Stranded Adeno-Associated Virus Achieves Efficient Gene Transfer to Anterior Segment in the Mouse Eye,” PLoS ONE 12(8):e0182473 (2017).

In some cases, the rAAV vector is of the serotype AAV1, AAV2, AAV4, AAV5, AAV6, AAV7, AAV8, AAV9, AAV10, AAV11, AAV12, AAV13, or Anc80L65. Anc80L65 is described in Sharma et al., “Transduction Efficiency of AAV 2/6, 2/8 and 2/9 Vectors for Delivering Genes in Human Corneal Fibroblasts,” PLoS ONE 12(8):e0182473 (2017). Production of pseudotyped rAAV is disclosed in, for example, WO 01/83692. Other types of rAAV variants, for example rAAV with capsid mutations, are also contemplated. See, for example, Marsic et al., Mol. Ther. 22(11):1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector. Other combinations of genome and serotype are contemplated by the present disclosure, including, without limitation, those described in Sharm et al., “Transduction Efficiency of AAV 2/6, 2/8 and 2/9 Vectors for Delivering Genes in Human Corneal Fibroblasts,” Brain Res. Bull. 2010 Feb. 15; 81(2-3):273.

### 2.5 Promoters

In some cases, a polynucleotide sequence encoding a therapeutic protein or a matrix metalloproteinase or MMP-3 is operatively linked to an inducible promoter. A polynucleotide sequence operatively linked to an inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). This list of inducible promoters is non-limiting.

In some embodiments, the polynucleotide sequence encoding matrix metalloproteinase 3 (MMP-3) is operably linked to a CMV promoter. The present disclosure further contemplates the use of other promoter sequences. Promoters useful in embodiments of the present disclosure include, without limitation, cytomegalovirus (CMV) and murine stem cell virus (MSCV), phosphoglycerate kinase (PGK), a promoter sequence comprised of the CMV enhancer and portions of the chicken beta-actin promoter and the rabbit beta-globin gene (CAG), promoter sequence comprised of portions of the SV40 promoter and CD43 promoter (SV40/CD43), and a synthetic promoter that contains the U3 region of a modified MoMuLV LTR with myeloproliferative sarcoma virus enhancer (MND). In some cases, the promoter may be a synthetic promoter. Exemplary synthetic promoters are provided by Schlabach et al., “Synthetic Design of Strong Promoters,” Proc. Natl. Acad. Sci. USA, 2010 Feb. 9; 107(6):2538-2543.

### 2.6 Polynucleotide Sequences of MMP-3 and Homologs

As used herein, the term “MMP-3” refers to the matrix metalloproteinase 3 encoded by the human genome, including any allelic variant thereof, as well as alternatively to a matrix metalloproteainase 3 from any other mammalian genome. It may be advantageous to match the MMP-3 to the subject to which the rAAV encoding that MMP-3 is administered. It will be understood, however, that an MMP-3 from another species may be suitable for use in a human subject, or that human MMP-3 may be used in treatment of another species of mammal, including, without limitation, a horse, a dog, a cat, a pig, or a primate. So long as the MMP-3 retains activity in the subject, the MMP-3 will be suitable for use in that subject.

The present disclosure also contemplates the use of sequence variants of MMP-3. In some cases, it may be advantageous to engineer the MMP-3 sequence to increase or decrease MMP-3 activity, to minimize immunogenicity, or to alter the pharmacokinetic properties of the MMP-3. In one aspect, described herein the polynucleotide sequence is a recombinant AAV vector comprising a polynucleotide sequence encoding MMP-3. The present disclosure provides a human MMP-3 polynucleotide sequence (SEQ ID: 1) and a mouse MMP-3 polynucleotide sequence (SEQ ID: 3). In some embodiments, the polynucleotide sequence encoding MMP-3 comprises a sequence is at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence set forth in SEQ ID NO: 1, or to the nucleotide sequence set forth in SEQ ID NO: 3, and encodes protein that retains MMP-3 activity. In some embodiments, the polynucleotide sequence encoding MMP-3 comprises the nucleotide sequence set forth in SEQ ID NO: 1. In some case, the polynucleotide sequence encoding MMP-3 consists the nucleotide sequence set forth in SEQ ID NO: 1. In another aspect, a recombinant AAV vector described herein comprises a polynucleotide sequence encoding MMP-3 that is at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% identical to the amino acid sequence of SEQ ID NO: 2, and the protein retains MMP-activity.

### 2.7 In Vivo and In Vitro Assays

The present disclosure further relates to assessment of efficacy and safety of gene therapy vectors in in vitro assay systems. The disclosure provides a recombinant AAV (rAAV) vector comprising a polynucleotide sequence encoding matrix metalloproteinase 3 (MMP-3). Using this rAAV vector or vectors delivering transgene for other therapeutic proteins, one can treat vision conditions such as glaucoma by administering the rAAV to the eye. In some cases, treatments aims to lower ocular pressure, and one means of achieving lower ocular pressure is through remodeling or degrading the extracellular matrix by the therapeutic protein, such as MMP-3 or the like. The effect can be assessed by measuring the permeability of the extracellular matrix of the trabecular meshwork of the eye or by measuring in an in vitro assay the effect of the rAAV. Suitable in vitro assays disclosed by the present inventions include use of human Schlemm's Canal (SC) endothelial cells (SCEC) monolayers derived from either human glaucomatous, primary open angle glaucoma (POAG) or control (cataract) cultured in aqueous humour (AH). Transendothelial electrical resistance (TEER) and permeability to a fluorescent-linked dye can then be measured in cells transduced with rAAV vector or not transduced for comparison. In other assays, ECM proteins can be stained and observed by immunofluorescence. These and other in vitro assays are described in more detail as follows.

Contacting the rAAV vector to a human trabecular meshwork (HTM) monolayer may increase the rate of tracer molecule flux through such a monolayer by more than about 5, 6, 7, 8, 9, 10, 11, 12, 13, or 15% over the tracer molecule flux through a HTM monolayer not contacted with said rAAV. As used herein, the terms “tracer molecule flux” or “tracer flux” refer to the flow of a tracer molecule across an epithelial membrane as described, for example, in Dawson et al., “Tracer Flux Ratios: A Phenomenological Approach,” J. Membr. Biol. 1977 Mar. 23; 31(4):351-358. Optionally, the tracer may be dextran conjugated to fluorescein isothiocyanate (FITC-dextran). In cases, contacting said rAAV vector to a human trabecular meshwork (HTM) monolayer decreases the transendothelial electrical resistance (TEER) of said monolayers by more than about 1, 2, 3, 4, 5, 6, 7, 8, 9, 10, 11, 12, 13, 14, or 15 Ohm per cm², more than about 15 Ohm per cm², or more than about 20 Ohm per cm² over the TEER of a monolayer not contacted with said rAAV. Methods of determining TEER are described in Srinivasan et al., “TEER Measurement Techniques for In Vitro Barrier Model Systems,” J. Lab. Autom. 2015 April; 20(2):107-126. doi:10.1177/2211068214561025. Epub 2015 Jan. 13.

In the eye of a subject, in vivo, administering the rAAV to the eye may, in some cases, increase permeability of the extracellular matrix of the trabecular meshwork, decrease outflow resistance of said eye, and/or decrease intraocular pressure (IOP). Measurement of outflow resistance and intraocular pressure of an eye is described in the examples that follow this detailed description, and in, for example, in Sherwood at al., “Measurement of Outflow Facility Using iPerfusion,” PLoS One, 2016, 11:e0150694.

The intraocular pressure (IOP) of a subject or a mammal to which a composition is administered may be decreased by more than 1, 2, 3, 4, or 5 mmHg. The outflow rate may be increased by more than 1, 2, 3, 4, 5, 10, or 15 nl/min/mmHg. The optically empty length in the trabecular meshwork of a subject or mammal may be increased by more than about 5, 10, 15, 20, 25, 30, 35, 40, 45, or 50%. Generally, rAAV vectors cause transduction of cells to which they are contacted. The transduced cells may be cells of the corneal endothelium, as well as other ocular cells. After administration, MMP-3 concentration in aqueous humor of is increased by about 0.1, 0.2, 0.3, 0.4, 0.5, or 0.6 ng/ml, or any value in between, such as in particular an increase of about 0.49 ng/ml or greater. In some embodiments, MMP-3 activity in aqueous humor of said eye is increased by about 1, 2, 3, 4, 5, or 6, mU or greater, or any value in between, such as in particular by about 5.34 mU or greater. It is further disclosed that the corneal thickness of said mammal is unchanged following treatment.

### 2.8 Therapeutic Compositions and Methods

As used herein, the term “patient in need” or “subject in need” refers to a patient or subject at risk of, or suffering from, a disease, disorder or condition that is amenable to treatment or amelioration with a rAAV comprising a nucleic acid sequence encoding matrix metalloproteinase or a composition comprising such a rAAV provided herein. A patient or subject in need may, for instance, be a patient or subject diagnosed with a disease associated with the malfunction of matrix metalloproteinase, such as glaucoma. A subject may have a mutation or a malfunction in a matrix metalloproteinase gene or protein. “Subject” and “patient” are used interchangeably herein.

The subject treated by the methods described herein may be a mammal. In some cases, a subject is a human, a non-human primate, a pig, a horse, a cow, a dog, a cat, a rabbit, a mouse or a rat. A subject may be a human female or a human male. Subjects may range in age, including juvenile onset glaucoma, early onset adult glaucoma, or age-related glaucoma. Thus, the present disclosure contemplates administering any of the rAAV vectors disclosed to a subject suffering from juvenile onset glaucoma, to a subject suffering from early onset adult glaucoma, or to a subject suffering from age-related glaucoma.

Combination therapies are also contemplated by the invention. Combination as used herein includes simultaneous treatment or sequential treatment. Combinations of methods of the invention with standard medical treatments (e.g., corticosteroids or topical pressure reducing medications) are specifically contemplated, as are combinations with novel therapies. In some cases, a subject may be treated with a steroid to prevent or to reduce an immune response to administration of a rAAV described herein. In certain cases, a subject may receive topical pressure reducing medications before, during, or after administrating of an rAAV described herein. In certain cases, a subject may receive a medication capable of causing the pupil of the eye to dilate (e.g., tropicamide and/or phenylephrine). In certain cases, the subject may receive a moisturizing gel during recovery to prevent corneal dehydration.

A therapeutically effective amount of the rAAV vector is a dose of rAAV ranging from about 1e13 vg/kg to about 5e14 vg/kg, or about 1e13 vg/kg to about 2e13 vg/kg, or about 1e13 vg/kg to about 3e13 vg/kg, or about 1e13 vg/kg to about 4e13 vg/kg, or about 1e13 vg/kg to about 5e13 vg/kg, or about 1e13 vg/kg to about 6e13 vg/kg, or about 1e13 vg/kg to about 7e13 vg/kg, or about 1e13 vg/kg to about 8e13 vg/kg, or about 1e13 vg/kg to about 9e13 vg/kg, or about 1e13 vg/kg to about 1e14 vg/kg, or about 1e13 vg/kg to about 2e14 vg/kg, or 1e13 vg/kg to about 3e14 vg/kg, or about 1×13 to about 4e14 vg/kg, or about 3e13 vg/kg to about 4e13 vg/kg, or about 3e13 vg/kg to about 5e13 vg/kg, or about 3e13 vg/kg to about 6e13 vg/kg, or about 3e13 vg/kg to about 7e13 vg/kg, or about 3e13 vg/kg to about 8e13 vg/kg, or about 3e13 vg/kg to about 9e13 vg/kg, or about 3e13 vg/kg to about 1e14 vg/kg, or about 3e13 vg/kg to about 2e14 vg/kg, or 3e13 vg/kg to about 3e14 vg/kg, or about 3e13 to about 4e14 vg/kg, or about 3e13 vg/kg to about 5e14 vg/kg, or about 5e13 vg/kg to about 6e13 vg/kg, or about 5e13 vg/kg to about 7e13 vg/kg, or about 5e13 vg/kg to about 8e13 vg/kg, or about 5e13 vg/kg to about 9e13 vg/kg, or about 5e13 vg/kg to about 1e14 vg/kg, or about 5e13 vg/kg to about 2e14 vg/kg, or 5e13 vg/kg to about 3e14 vg/kg, or about 5e13 to about 4e14 vg/kg, or about 5e13 vg/kg to about 5e14 vg/kg, or about 1e14 vg/kg to about 2e14 vg/kg, or 1e14 vg/kg to about 3e14 vg/kg, or about 1e14 to about 4e14 vg/kg, or about 1e14 vg/kg to about 5e14 vg/kg. The invention also comprises compositions comprising these ranges of rAAV vector.

For example, a therapeutically effective amount of rAAV vector is a dose of 1e13 vg/kg, about 2e13 vg/kg, about 3e13 vg/kg, about 4e13 vg/kg, about 5e13 vg/kg, about 6e13 vg/kg, about 7e13 vg/kg, about 8e13 vg/kg, about 9e13 vg/kg, about 1e14 vg/kg, about 2e14 vg/kg, about 3e14 vg/kg, about 4e14 vg/kg and 5e14 vg/kg. The invention also comprises compositions comprising these doses of rAAV vector.

In some cases, the therapeutic composition comprises more than about 1e9, 1e10, or 1e11 genomes of the rAAV vector per volume of therapeutic composition injected. In some cases, the therapeutic composition comprises more than approximately 1e10, 1e11, 1e12, or 1e13 genomes of the rAAV vector per mL.

### 2.8 Administration of Compositions

Administration of an effective dose of the compositions may be by routes standard in the art including, but not limited to, intracameral inoculation, intravitreal inoculation, subretinal inoculation, suprachroidal inoculation, canaloplasty, or episcleral vein-mediated delivery. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the matrix metalloproteinase.

The disclosure provides for local administration and systemic administration of an effective dose of rAAV and compositions of the invention. For example, systemic administration is administration into the circulatory system so that the entire body is affected. Systemic administration includes enteral administration such as absorption through the gastrointestinal tract and parental administration through injection, infusion or implantation. Systemic administration includes injection into the episcleral vein in order to transduce Schlemm's Canal endothelium with rAAV.

In particular, actual administration of rAAV of the present invention may be accomplished by using any physical method that will transport the rAAV recombinant vector into the target tissue of an animal. Administration according to the invention includes, but is not limited to, injection into the bloodstream and/or directly into the eye. Simply resuspending a rAAV in phosphate buffered saline has been demonstrated to be sufficient to provide a vehicle useful for eye expression, and there are no known restrictions on the carriers or other components that can be co-administered with the rAAV (although compositions that degrade DNA should be avoided in the normal manner with rAAV).

Capsid proteins of a rAAV may be modified so that the rAAV is targeted to a particular target tissue of interest such as eye. See, for example, WO 02/053703, the disclosure of which is incorporated by reference herein. Pharmaceutical compositions can be prepared as injectable formulations or as topical formulations to be delivered to the eyes by administration of eye drops or otherwise. Additionally, when a tetracycline-inducible promoter is used to control transgene expression, it may be advantageous to co-administer doxycycline via eyedrops. Numerous formulations of rAAV have been previously developed and can be used in the practice of the invention. The rAAV can be used with any pharmaceutically acceptable carrier for ease of administration and handling.

For purposes of injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target cells are removed from the subject, transduced with rAAV and reintroduced into the subject. Alternatively, syngeneic or xenogeneic ocular cells can be used where those cells will not generate an inappropriate immune response in the subject.

Suitable methods for the transduction and reintroduction of transduced cells into a subject are known in the art. In one embodiment, cells can be transduced in vitro by combining rAAV with cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intracameral inoculation, intravitreal inoculation, subretinal inoculation, canaloplasty, or episcleral vein-mediated delivery. Transduction of cells with rAAV of the invention results in sustained expression of matrix metalloproteinase. The present invention thus provides methods of administering/delivering rAAV which express matrix metalloproteinase to a mammalian subject, preferably a human being. These methods include transducing tissues (including, but not limited to, the tissues of the eye) with one or more rAAV of the present invention. Transduction may be carried out with gene cassettes comprising tissue specific control elements. For example, one embodiment of the invention provides methods of transducing eye cells and eye tissues directed by eye specific control elements, including, but not limited to, those derived from corneal endothelia or Schlemm's Canal endothelium enriched promoters, and other control elements.

The invention is further described in the following Examples, which do not limit the scope of the invention described in the claims.

### EXAMPLES

### Example 1: Effects of Glaucomatous Aqueous Humor on SC Endothelial and TM Cell Monolayers

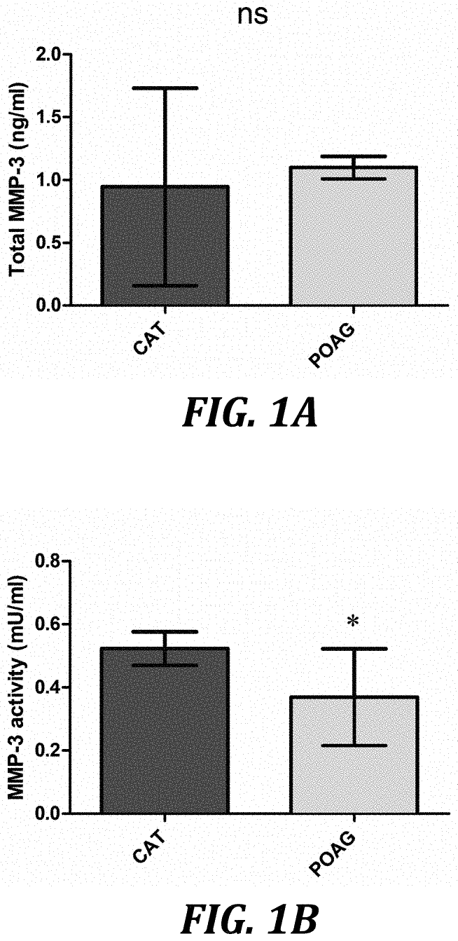

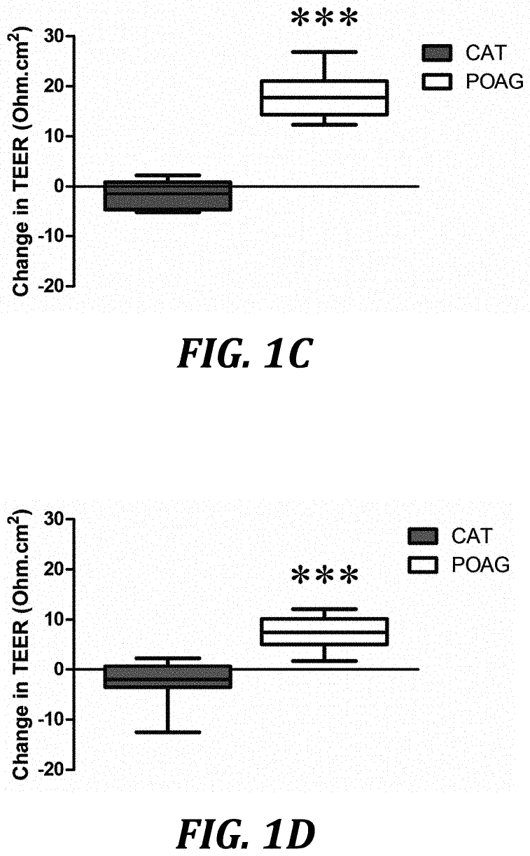

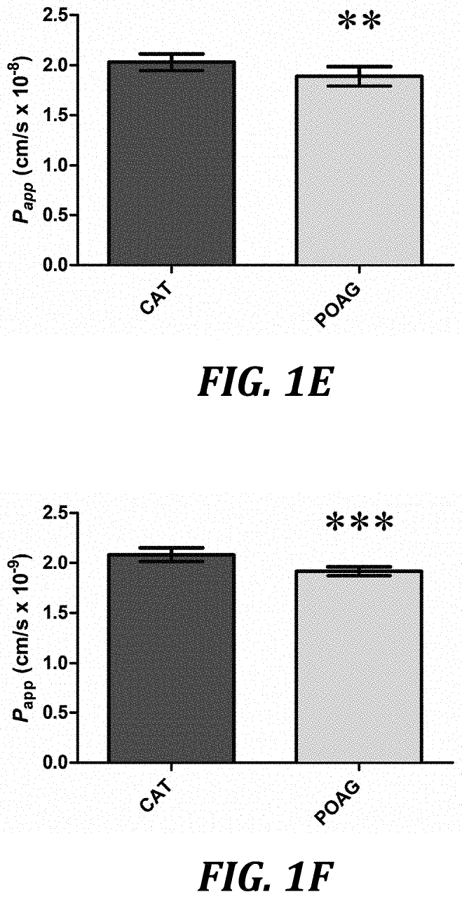

The present inventors treated cultured human SCEC monolayers with human glaucomatous (POAG) or control (cataract) AH for 24 h, and quantified levels of total secreted and activated MMP-3 in culture media. This was achieved by performing an ELISA and FRET assay, to monitor the degree of cleavage of an MMP-3 specific substrate, on cell media 24 h post-treatment. The present inventors did not observe a significant increase in the level of total (latent and active forms) secreted MMP-3 in culture media following treatment with POAG aqueous, with an increase of 0.15 [−0.35, 0.66] ng/ml (mean [95% confidence interval (CI)]) (P=0.45, n=3, FIG. 1A) over controls. However, activity assays indicated that the MMP-3 secreted in response to POAG aqueous had less enzymatic activity than that of cataract control AH, with an average change of −0.15 [−0.28, −0.02] mU/ml (P=0.024, n=9 cataract, n=7 POAG, FIG. 1B). These observations corroborate results obtained involving other members of the MMP family in POAG aqueous in that the amount of secreted MMP may remain relatively unchanged, but its proteolytic activity is reduced.

Effects of glaucomatous AH on the permeability of SCEC and human TM (HTM) monolayers were determined by transendothelial electrical resistance (TEER) and FITC-dextran flux assays. Treatment of cultured SCEC monolayers with POAG AH resulted in increased TEER by an average of 102% after 24-h treatment compared to control AH (−7%), displaying an average absolute increase of 19.82 [15.82, 23.81] Ohm·cm² (P<0.0001, n=6 cataract, n=12 POAG, FIG. 1C). Similarly, HTM responded with an increase of 9.79 [5.55, 14.05] Ohm·cm² in response to glaucomatous AH, (P=0.0002, n=8, FIG. 1D). Glaucomatous AH also reduced par-acellular flux, as measured by permeability co-efficient (Papp), to dextran of 70 kDa as compared to cataract controls, with a mean difference of 0.14 [0.05, 0.22] cm/s×10⁻⁸ (P=0.009, n=3 cataract, n=3 POAG, FIG. 1E). A reduction in HTM permeability was also observed with a mean difference of 0.17 [0.09, 0.23] cm/s×10⁻⁹ (P=0.005, n=8 cataract, n=7 POAG, FIG. 1F).

### Example 2: Treatment of Outflow Cell Monolayers with Recombinant Human MMP-3 Increases Permeability with Concomitant Reductions in TEER

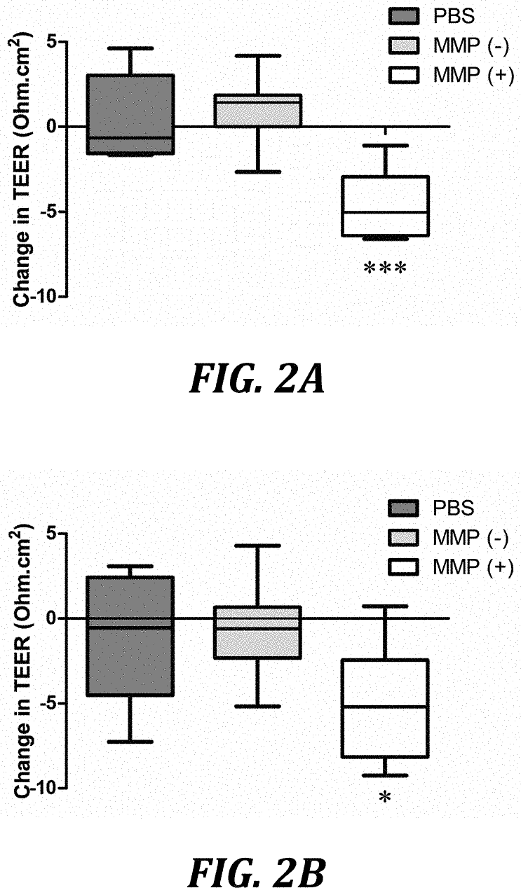

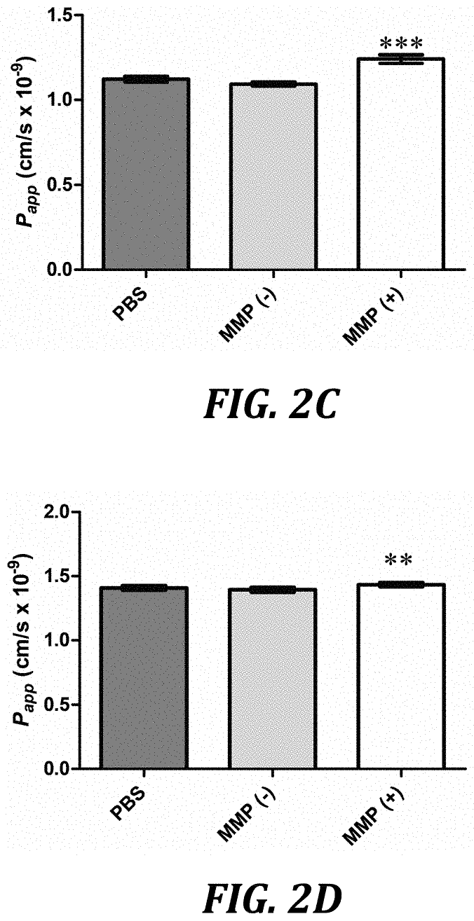

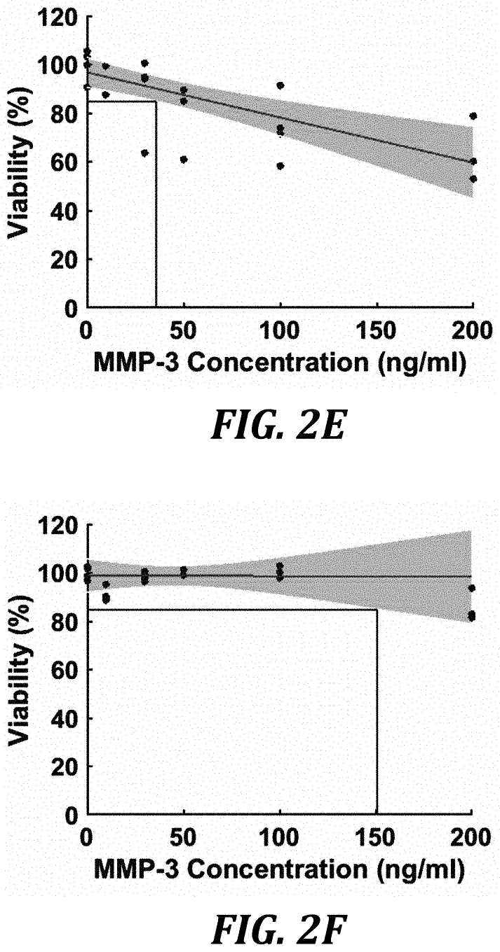

In contrast to the negative effects of glaucomatous AH on SCEC and HTM permeability and resistance, we observed that treatment of cultured monolayers with 10 ng/ml of active recombinant human MMP-3 (SEQ ID NO: 3) reduced TEER values on average by 5.62 [2.92, 8.32] Ohm·cm² greater than inactivated MMP-3 controls over the course of 24 h for SCEC (P<0.0001, n=8, FIG. 2A) and by 4.29 [0.11, 8.48] Ohm·cm² for HTM (P=0.0137, n=8, FIG. 2B) respectively. Permeability assays complemented these data as increases in paracellular flux of 70 kDa FITC-dextran by 0.14 [0.12, 0.18] cm/s×10⁻⁹ (P<0.0001, n=8, FIG. 2C) were observed in SCEC, and 0.04 [0.01, 0.06] cm/s×10⁻⁹ (P<0.01, n=8, FIG. 2D) in HTM monolayers when comparing treatments of MMP-3 to its inactivated counterpart control: TIMP-1 incubated with MMP-3. To rule out cytotoxicity as a reason for the observed changes in paracellular permeability, a cell viability assay was undertaken. Based on data shown in FIG. 2E, for concentrations below 36 ng/ml MMP-3, the average SCEC cell viability for n=3 will exceed 85%. Greater tolerability was observed in HTM cases, retaining an average viability of at least 85% for MMP-3 concentrations up to 151 ng/ml (n=3, FIG. 2F).

### Example 3: Treatment of SCEC and HTM Monolayers with Active Recombinant Human MMP-3 Induces Remodeling and Degradation of ECM Components

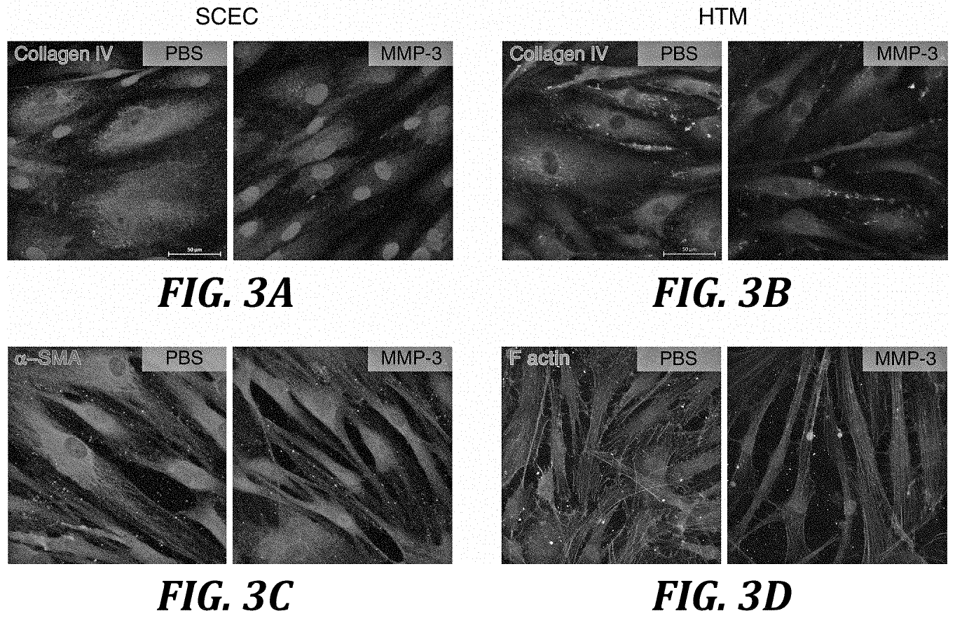

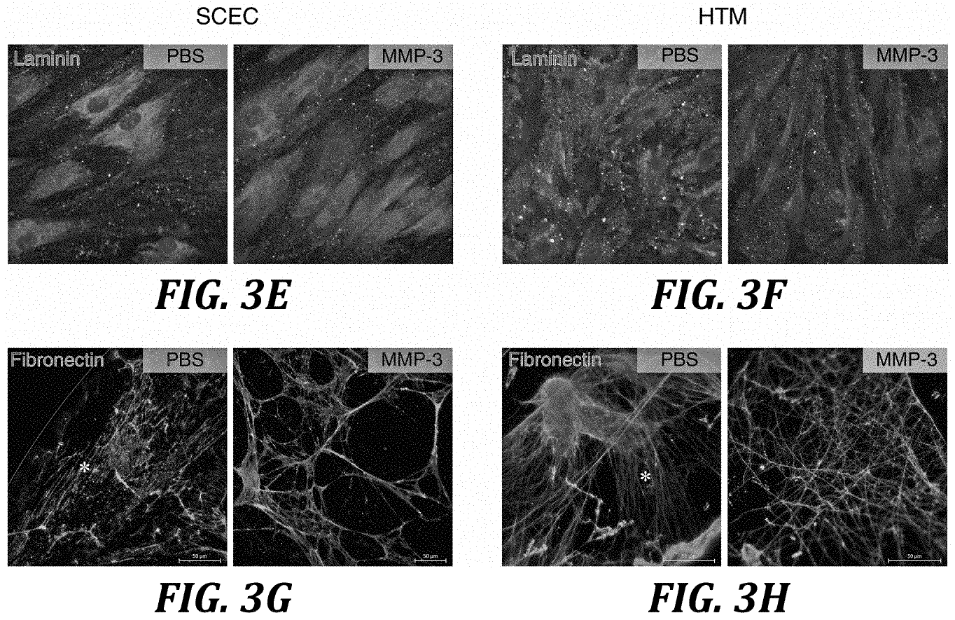

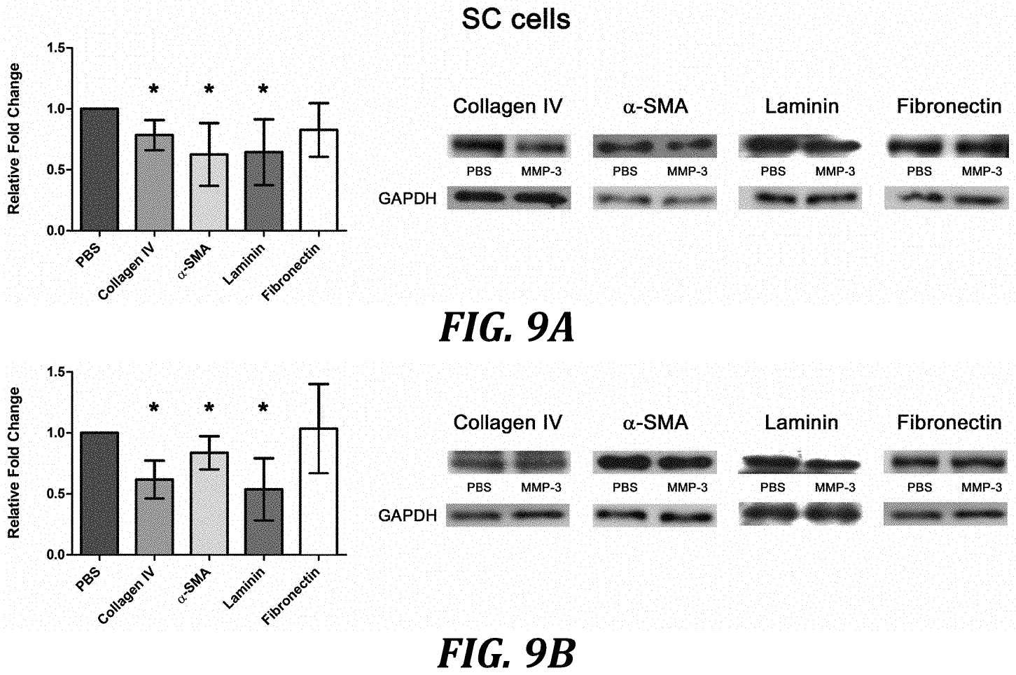

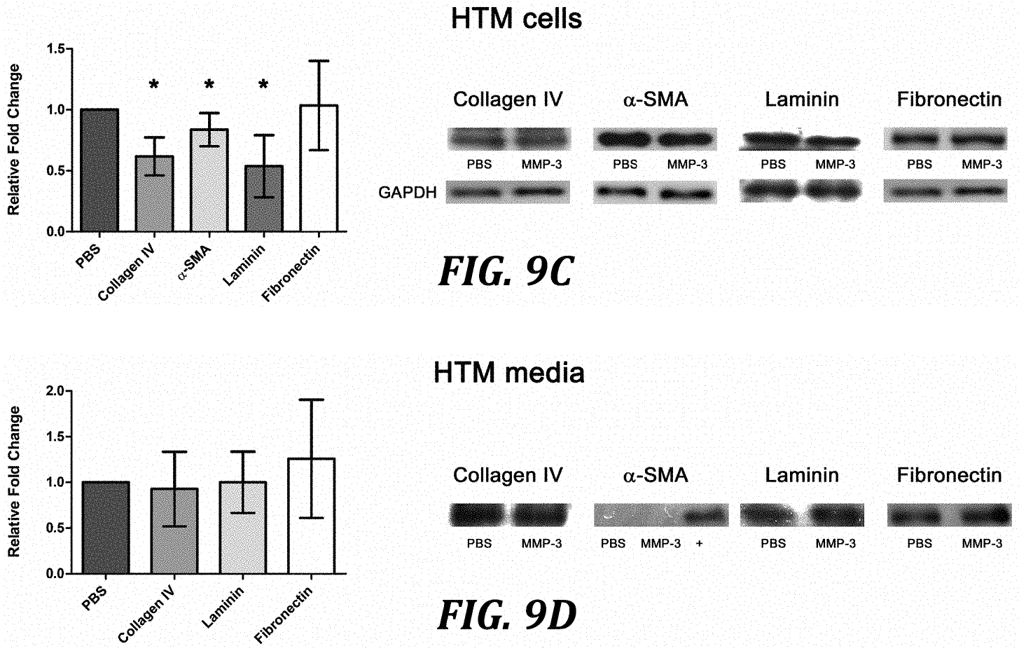

In order to attribute increases in permeability to the ECM remodeling effects associated with MMP-3, SCEC and HTM monolayers were both treated as above with 10 mg/ml MMP-3 for 24 h. Following treatment, we observed changes in the staining pattern and intensity of a number of ECM proteins by immunocytochemistry. Specific collagen IV staining was localized to perinuclear areas and cytoplasm in both SCEC and HTM cells (FIGS. 3A-3B). In particular, we observed a decrease in the staining intensity around perinuclear areas in treated cells as compared to controls. α-SMA fibers facilitating cell-cell contacts in SCEC localized specifically to the cytoplasm and cytoskeleton, and MMP-3 treatment led to an attenuation of fiber bundles with thinning of intercellular connections (FIG. 3C). Fluorescent images of F-actin in HTM monolayers also revealed constricted actin bundles and a reduced tendency for bundle crossovers (FIG. 3D). Immunofluorescence staining of laminin in SCEC and HTM cells showed diminished cytoplasmic localization and reduced network complexity and multiplicity in MMP-3 treated cells as compared to control staining intensity of laminin (FIGS. 3E-3F). To visualize fibronectin clearly without cellular interference, decellularization was performed after MMP-3 treatment to isolate the ECM scaffold from the cell monolayer. Fluorescent images show significant perturbation of fibronectin network in treated cells as opposed to the linear cellular organization observed in control cells (asterisk, FIGS. 3G-3H). To quantitatively demonstrate remodeling of these proteins, Western blot analysis was performed on both cell lysate and media fractions of SC and HTM cell monolayers (FIGS. 9A-9D).

Specific bands were observed at 300 kDa for collagen IV, 42 kDa for α-SMA, 220 kDa for laminin and 290 kDa for fibronectin. A significant reduction of collagen IV (P=0.01, P=0.01), α-SMA (P=0.04, P=0.04), and laminin (P=0.04, P=0.03) were observed in SC and HTM whole cell lysate samples respectively (n=4 for all cases). Collectively, these data clearly illustrate that MMP-3 mediates remodeling of ECM components in both SCEC and HTM cell monolayers.

### Example 4: Intracameral Inoculation of AAV-2/9 Expressing a CMV-Driven MMP-3 Gene Efficiently Transduces Corneal Endothelium and Results in Elevated Levels of MMP-3 in Aqueous Humor

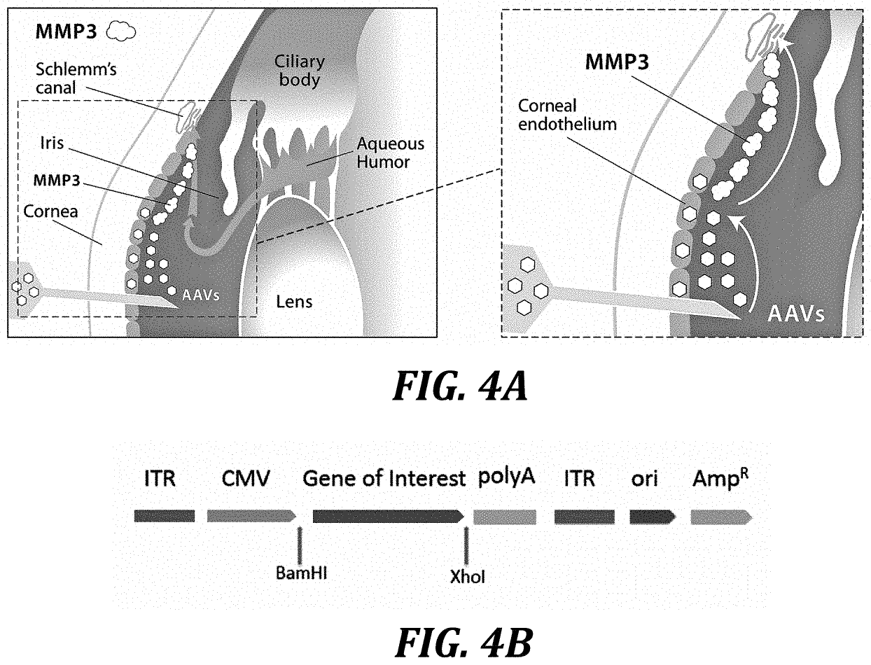

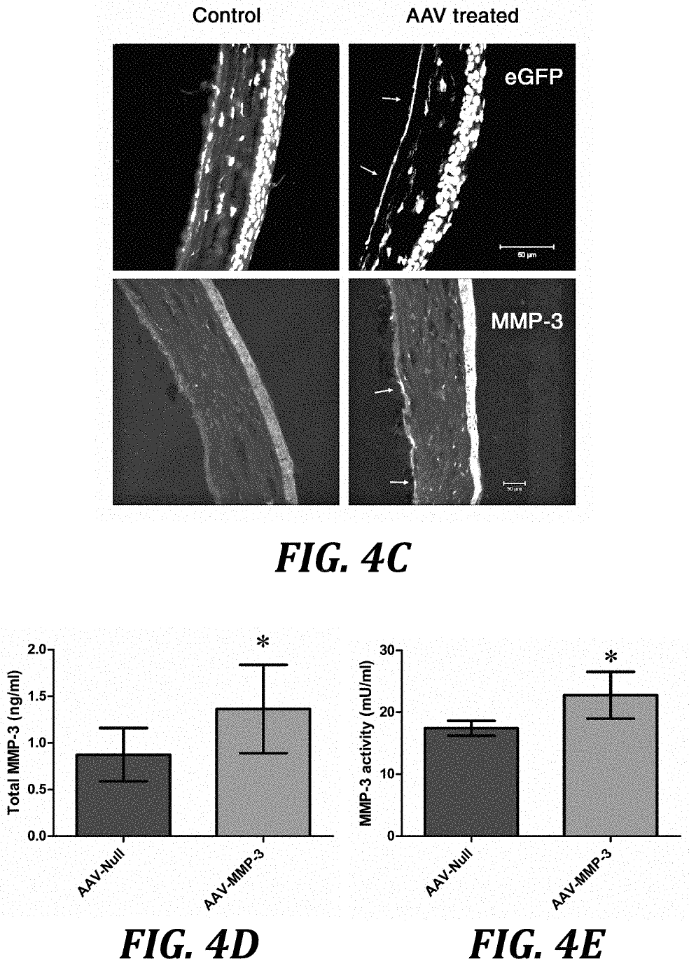

AAV-mediated transduction of corneal endothelium could, in principle, serve as an efficient means of expressing and secreting MMP-3 into AH. The advantage of such an approach is that the natural flow dynamics of AH will allow transportation of secreted MMP-3 towards the outflow tissues (FIG. 4A). We evaluated the efficiency of a number of AAV serotypes with either single stranded or self-complementary genomes to deliver MMP-3 to the outflow tissues. 2 μl of viral particles (2×10¹² vector genomes/ml) of each serotype, expressing a CMV-driven eGFP reporter gene (FIG. 4B) were intracamerally inoculated into wild type C57BL/6 mice and eyes examined via fluorescent microscopy at 3 weeks post-inoculation. Extensive expression of the reporter gene was observed in the corneal endothelium of eyes injected with non self-complementary AAV-2/9 (FIG. 4C top), with no fluorescence being detectable in the outflow tissues themselves using this construct. Hence, the eGFP cDNA from AAV-2/9 was exchanged with murine MMP-3 cDNA (SEQ ID NO: 3) to generate AAV-MMP-3, and similar inoculation resulted in MMP-3 (SEQ ID NO: 4) expression that was prominently detected in the corneal endothelium and not in null controls (FIG. 4C, bottom). No significant difference in central corneal thickness was detected following AAV inoculation between treated (116.7 [112.5, 120.9] μm) and control eyes (116.4 [113.6, 119.1] μm) (n=4). Corneas also appeared clear with no signs of cataracts upon visual inspection.

The level of total MMP-3 in the AH of twelve inoculated animals was quantified using enzyme-linked immunosorbent assay (ELISA), and we observed a significant average increase in total MMP-3 protein of 56%, 1.37 [0.89, 1.84] ng/ml as compared to 0.87 [0.59, 1.12] ng/ml for control AAV (P=0.016, n=12, FIG. 4D). The activity of AAV-mediated production of MMP-3 was also assessed using FRET, and a significant increase in activity of 34 [6.86, 61.14] % was observed, on average, in AAV-MMP-3 treated eyes compared to contralateral controls (P=0.0164, n=17, FIG. 4E).

### Example 5: Intracameral Inoculation of AAV-2/9 Expressing an MMP-3 Gene Increases Outflow Facility and Reduces IOP in Murine Eyes

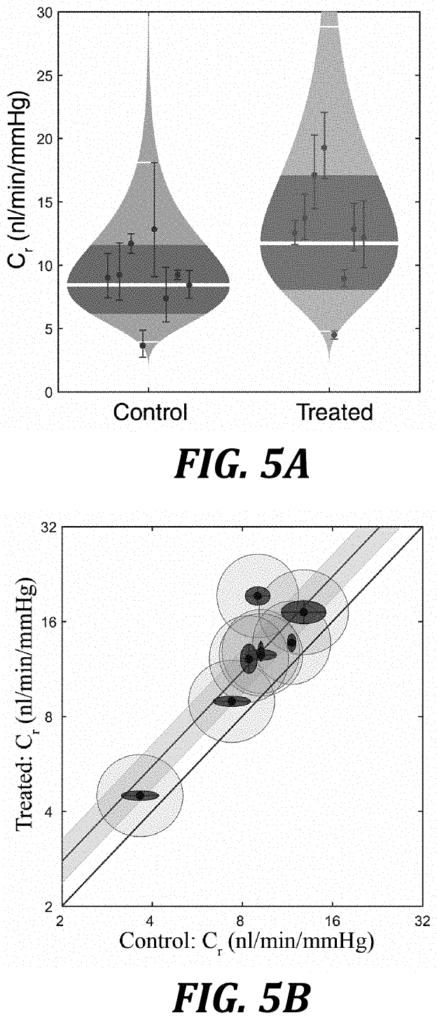

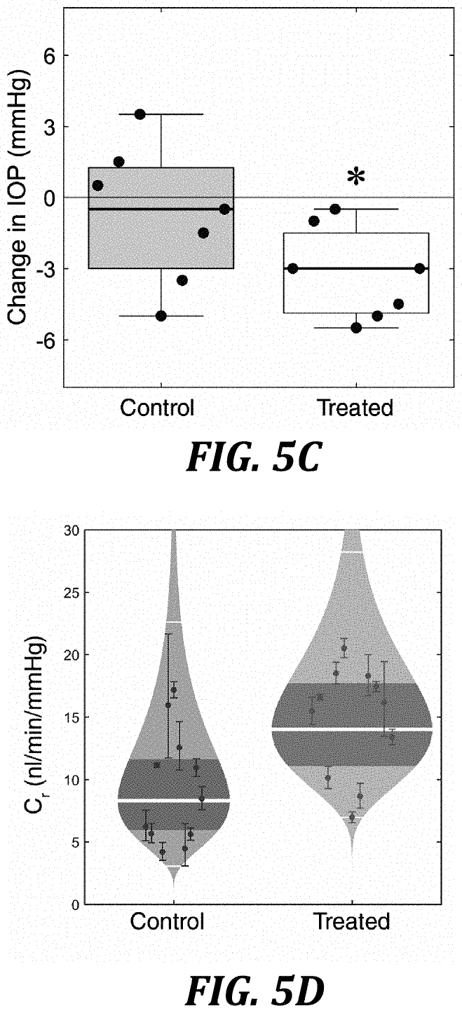

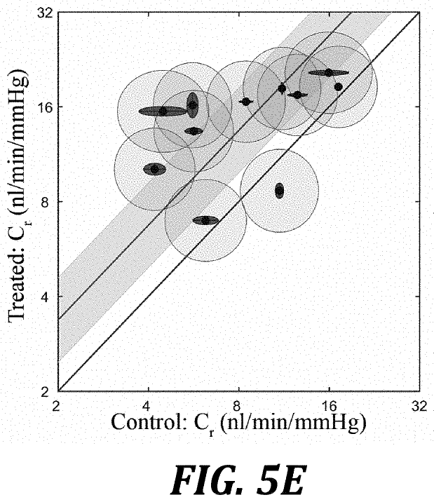

In order to determine the effect of AAV-mediated expression of MMP-3 from the corneal endothelium on aqueous outflow, the conventional outflow facility was measured using the recently developed iPerfusion™ system designed specifically to measure conventional outflow facility in mice. See Sherwood, J. M., Reina-Torres, E., Bertrand, J. A., Rowe, B. and Overby, D. R, “Measurement of Outflow Facility Using iPerfusion,” PLoS One, 11, e0150694 (2016). Wild type mice were intracamerally injected with 1×10¹¹ vector genomes of AAV-MMP-3, and contralateral eyes received the same quantity of AAV-Null. Four weeks post-inoculation, eyes were enucleated and perfused in pairs over incrementing steps in applied pressure. The resulting facility data presented in FIGS. 5A and 5B clearly illustrate that control eyes have an average facility of 8.44 [6.14, 11.60] nl/min/mmHg, with treated eyes having an average facility of 11.73 [8.05, 17.08] nl/min/mmHg. There is, therefore, an average increase in outflow facility of 39 [19, 63] % in pairs, between treated eyes and their contralateral controls (P=0.002, n=8 pairs).

As the major pathology in POAG is IOP elevation, and an increased outflow facility was observed, tonometric IOP measurements were taken both immediately before (pre), and four weeks after (post) intracameral injection of AAV-2/9 expressing MMP-3 (SEQ ID NO: 3) or a null vector in the case of the control. Differences between pre- and post-injection IOP were calculated using the non-parametric Wilcoxon matched-pairs signed rank test. Eyes treated with AAV-Null had no significant change in IOP −0.5±2.9 mmHg (median±median absolute deviation (MAD), P ¼ 0.61, n ¼ 7, Wilcoxon signed-rank test with a theoretical median IOP change of 0) after treatment. In comparison, when treated with AAV-MMP-3, median IOP significantly decreased by 3.0±2.9 mmHg (P=0.022, n=7, FIG. 5C). The IOP difference in AAV-MMP-3 treated eyes was significantly greater than the IOP difference in the contralateral AAV-Null treated eyes by 2.5±0.7 mmHg (P=0.034, n=7, FIG. 5C).

### Example 6: Controlled Periodic Activation of MMP-3

To incorporate a control mechanism for the secretion of MMP3 from corneal endothelium, we first introduced AAV-2/9 expressing eGFP under the control of a tetracycline-inducible promoter into the anterior chambers of both eyes of wild type mice. After 3 weeks, mice were treated with a regime of one drop of 0.2% doxycycline (a tetracycline derivative) two times per day (approx. 8 h between each application) for 10-16 days in one eye only. PBS was administered onto the contralateral eye as a control. Extensive expression of the reporter gene was observed only in the corneal endothelium, and no expression was observed in the contralateral control. Following this, we replaced the reporter cDNA with murine MMP-3 cDNA and the resulting AAV (Induc. AAV-MMP-3) was injected into the anterior chambers of animals at 1×10¹¹ viral genomes per eye. Using the inducible eGFP virus (Induc. AAV-eGFP) as a contra-lateral control, expression was induced by administering doxycycline (as above) to both eyes. Contralateral eyes were perfused as above, the control group exhibiting an average facility of 8.30 [5.75, 11.26] nl/min/mmHg and the MMP-3 treatment group resulting in a facility of 14.01 [11.09, 17.72] nl/min/mmHg. Paired, these eyes exhibit an average increase in outflow facility of 68 [24, 128] % (P=0.004, n=11, FIGS. 5D and 5E). This observation strongly supports the concept that MMP-3 expression could be induced in a controlled and reversible manner, with periodic IOP measurements utilized to guide the induction of expression.

### Example 7: Ultrastructural Analysis of AAV-MMP-3 Treated Eyes

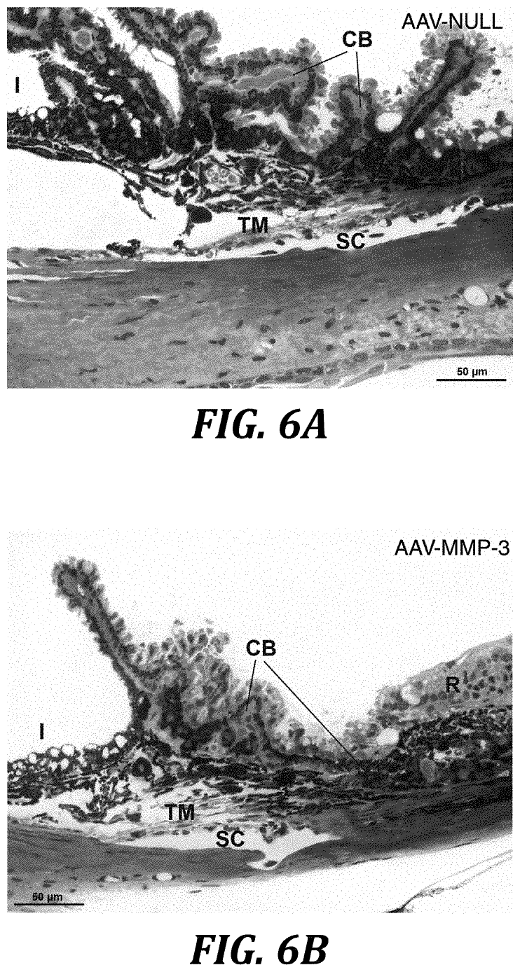

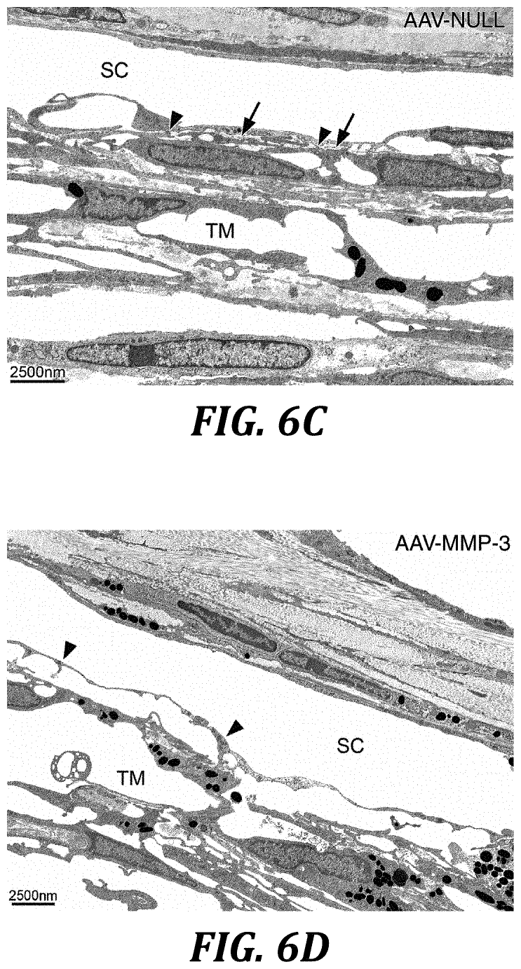

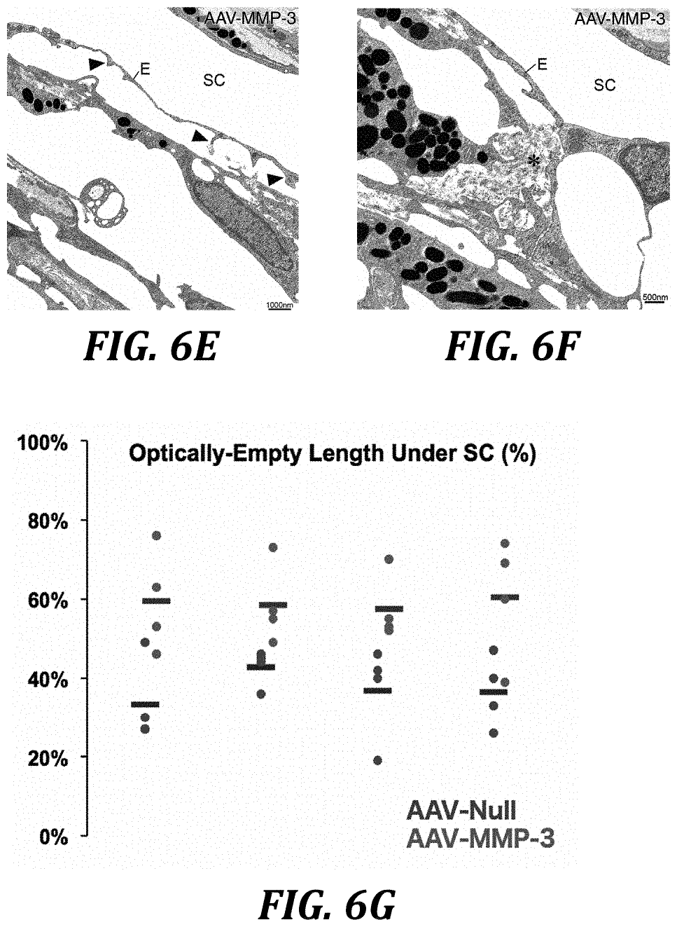

In order to evaluate whether the AAV-MMP-3 treatment affects the morphology of the eye and the TM including the inner wall of SC, ultrastructural investigation was performed in four pairs of mouse eyes. Corneas appeared translucent and healthy on visual inspection during enucleation. Semi-thin sections clearly demonstrated that there were no signs of an inflammatory reaction, either in the TM or in the cornea, uvea or retina (FIGS. 6A and 6B). Ultrastructural analysis of control eyes revealed normal outflow structural morphology, cell-matrix attachments and cell-cell connections between the SC and TM. The inner wall endothelial cells formed foot-like connections with sub-endothelial TM cells, as well as connections to underlying elastic fibers and discontinuous basement membrane (FIG. 6C). However, in some regions of treated eyes, especially those with a prominent SC lumen and scleral spur-like structure typical of the nasal quadrant, there appeared to be more optically empty space directly underlying the inner wall endothelium of SC, compared to AAV-Null controls (FIG. 6D). In these optically empty spaces, foot-like extensions of the inner w all to the sub-endothelial layer were absent or disconnected from the sub-endothelial cells or elastic fibers (FIGS. 6D and 6E). Occasionally, we observed an accumulation of ECM clumps beneath the inner wall that were not observed within the controls (FIG. 6F) and may represent remnants of digested material.

We quantified the optically empty length directly underlying the inner wall of SC. In control eyes, the % optically empty length in any one region ranged from 19 to 49% with an average of 37%. In the treated eyes, the equivalent range was 39-76% with an average of 59% (FIG. 6G). The differences between control and experimental eyes for each pair ranged from 16 to 26%, which corresponded to a statistically significant increase in the proportion of open space underlying the inner wall with AAV-MMP-3 relative to AAV-Null (P=0.002, n=4; paired Student's t-test). These data indicate that reduced ECM material in the TM and along the inner wall of SC is associated with AAV-MMP-3 treatment and may explain the enhanced outflow facility and IOP reduction. Furthermore, these morphological changes, because they were absent from controls, could not be attributed to an inflammatory or lytic response to AAV alone.

### Example 8: Materials and Methods

Cell Culture

Human SCEC were isolated, cultured and fully characterized according to previous protocols. Briefly, cells were isolated from the SC lumen of human donor eyes using a cannulation technique. Isolated cells were tested for positive expression of VE-cadherin and fibulin-2, but absence of myocilin induction upon treatment with 100 nM dexamethasone for 5 days. Confluent cells displayed a characteristic linear fusiform morphology, were contact inhibited and generated a net transendothelial electrical resistance (TEER) greater than 10 Ω·cm². TEER values were confirmed again prior to MMP-3 treatments. SCEC strains used were SC82 and SC83 between passages 2 and 7. Dulbecco's modified eagle medium (Gibco, Life Sciences®) 1% Pen/Strep/glutamine (Gibco, Life Sciences®) and 10% foetal bovine serum (FBS) performance plus (Gibco, Life Sciences®) was used as culture media in a 5% CO₂ incubator at 37° C. Cells were passaged with trypsin-EDTA (Gibco-BRL®) and seeded into 12 well or 24 well transwell plates (Costar™, Corning®). Human trabecular meshwork (HTM) cells were isolated and fully characterized according to the procedures described in Stamer et al., Curr. Eye Res., 14:611-617 (1995); Stamer et al., Curr. Eye Res., 14:1095-1100 (1995); Stamer et al., Invest. Ophthalmol. Vis. Sci., 37:2426-2433 (1996). TM tissue is removed from human donor eyes using a blunt dissection technique, and TM cells are dissociated from the tissue using a collagenase digestion protocol as previously described. Isolated cells are characterized by their dramatic induction of myocilin protein following treatment with dexamethasone (100 nM) for 5 days as detailed before. HTM123 and HTM134 cells were cultured similar to SCEC's and matured for one week in 1% FBS media prior to treatment.

Human AH samples (detailed below) were added 1:10 to fresh media for cellular treatment for use with TEER and permeability assays as described below.

Recombinant human active MMP-3 (ab96555, Abcam®) was added to cell media at a concentration of 10 ng/ml for TEER, permeability assays, Western blotting and immunocytochemistry as described below. Inactivated MMP-3 controls were achieved by incubating active MMP-3 (10 ng/ml) with recombinant human active TIMP-1 (100 ng/ml, ab82104, Abcam®) in cell media for 1 h prior to treatment.

Animals

Animals and procedures used in this study were carried out in accordance with regulations set out by The Health Products Regulatory Authority (HPRA), responsible for the correct implementation of EU directive 2010/63/EU. 8-11-week-old male and female C57BL/6 mice were used in all experimentation outlined in this study. Animals were bred and housed in specific-pathogen-free environments in the University of Dublin, Trinity College and all injections and IOP measurements complied with the HPRA project authorization number AE19136/P017.

Patient Aqueous Humor Samples

Human aqueous was obtained from the Mater Misericordiae Hospital, Dublin, Ireland. Upon informed consent, AH samples were collected from both POAG and control patients undergoing routine cataract surgery. The criteria for POAG was defined as the presence of glaucomatous optic disc cupping with associated visual field loss in an eye with a gonioscopically open anterior drainage channel, with an intraocular pressure >21 mmHg. The samples were taken immediately prior to corneal incision at the start of the procedure using a method described previously. Human AH collection conformed to the WMA Declaration of Helsinki and was approved by the Mater Misericordiae University Hospital Research Ethics Committee.

TEER Measurement

Electrical resistance values were used as a representative of the integrity of the endothelial cell-cell junctions. Cells grown on Costar transwell-polyester membrane inserts with a pore size of 0.4 μm were treated with 10 ng/ml MMP-3 as described above. TEER readings were measured before and 24 h after treatment. An electrical probe (Millicell ERS-2™ Voltohmmeter, Millipore®) was placed into both the apical and basal chambers of the transwells and a current was passed through the monolayers, reported as a resistance in Ω·cm². A correction was applied for the surface area of the membrane (0.33 cm²) and for the electrical resistance of the membrane (blank transwell).

Permeability Assessment by FITC-Dextrain Flux

The extent of monolayer permeability was assessed by the basal to apical movement of a tracer molecule through the mono-layer. Measures of permeability were taken 24 h after treatment immediately after TEER values, keeping experimental set-up identical to that of TEER readings. The permeability protocol was repeated as described in Keaney et al., “Autoregulated Paracellular Clearance of Amyloid-Beta Across the Blood-Brain Barrier,” Sci. Adv., 1, e1500472 (2015). A 70 kDa fluorescein isothiocyanate (FITC)-conjugated dextran (Sigma®) was added to the basal compartment of the transwell. Fresh medium was applied to the apical chamber and aliquots of 100 μl were taken every 15 min for a total of 120 min, replacing with fresh media. Sample aliquots were analyzed for FITC fluorescence (FLUOstar OPTIMA™, BMG Labtech®) at an excitation wavelength of 492 nm and emission wavelength of 520 nm. Relative fluorescent units (RFU) were converted to their corresponding concentrations by interpolating from a known standard curve. Corrections were made for background fluorescence and the serial dilutions generated over the experiments time course. Papp values were calculated representing the apparent permeability coefficient for control (PBS) and treatment (10 ng/ml MMP-3). This was achieved via the following equation:

Pₐₚₚ(cm/s)=(dM/dT)/(A×C₀),

Where dM/dT is the rate of appearance of FITC-dextran (FD) (μg/s) in the apical chamber from 0 to 120 min after the introduction of FD into the basal chamber. A is the effective surface area of the insert (cm²) and C₀ is the initial concentration of FD in the basal chamber.

Cell Viability

Cultured cells were treated with increasing concentrations of recombinant human MMP-3 (ab96555, Abcam®) from 0 to 200 ng/ml. Cell viability was assessed 24 h post-treatment with MMP-3 using a CellTitre 96® AQueous One Solution™ Cell Proliferation Assay (Promega®). Cell media was aspirated and a 1 in 6 dilution of the supplied reagent in media was added to the cell surface. Cells were incubated at 37° C. for 1 h and the media/reagent was transferred to a 96-well plate for reading by spectrophotometry (Multiskan FC™, Thermo Scientific®) at 450 nm. Standard in vitro viability calculations fail to consider sample size and the biological significance of the data. Hence, a modified approach was taken to determine at which concentration SCEC's show a reduced tolerability to MMP-3. This was defined at an average of 85% viability over three cell samples. This conservative value ensures that a cell population would remain viable and still be able to proliferate. Anything lower should be regarded as MMP-3 intolerability, i.e., reduced cell proliferation or cell death. Control samples (0 ng/ml MMP-3) were normalized to 100% viability and a linear model fitted to the normalized data. The MMP-3 concentration at which cells had an average of 85% viability was interpolated from the lower 95% confidence bound from this linear model. This value represents the concentration of MMP-3 at which the average of three cell samples would have a 97.5% chance of retaining a greater to or equal than 85% viability.

Immunocytochemistry (Cell Monolayers)

Immunocytochemistry was performed to visualize changes in ECM composition in response to MMP-3. Human SCEC and HTM were grown on chamber slides (Lab-Tek II®) and fixed in 4% paraformaldehyde (pH 7.4) for 20 min at room temperature and then washed with PBS for 15 min. Cell monolayers were blocked in PBS containing 5% normal goat serum (Ser. No. 10/658,654, Fischer Scientific®) and 0.1% Triton X-100 (T8787, Sigma®) at room temperature for 30 min. Primary antibodies of collagen IV (ab6586, Abcam), α-SMA (ab5694, Abcam®), laminin (ab11575, Abcam®) and F-actin (A12379, ThermoFisher Scientific®) were diluted at 1:100 in blocking buffer and incubated overnight at 4° C. Secondary antibodies (ab6939, Abcam®) were diluted at 1:500 in blocking buffer and then incubated for 2 h at room temperature. Following incubation, chamber slides were mounted with aquapolymount (Polyscience®) after nuclei-counterstaining with DAPI. Fluorescent images of SCEC monolayers were captured using a confocal microscope (Zeiss® LSM 710), and processed using imaging software ZEN 2012 (Zeiss®).

For clear fibronectin (ab23750, Abcam®) staining, cells were grown on cover slips and subsequently decellularized, leaving only the ECM material. Round cover slips (15 mm Diameter, Sparks Lab Supplies®) were silanized before cell seeding to enhance binding to ECM products. This was achieved by initially immersing slips in 1% acid alcohol (1% concentrated HCL, 70% ethanol, 29% dH₂O) for 30 mins. Slips were washed in running water for 5 min, immersed in dH₂O twice for 5 min, immersed in 95% ethanol twice for 5 min and let air dry for 15 min. Cover slips were then immersed in 2% APES (3-aminopropyl triethoxysilane (A3648, Sigma®) in acetone (Fisher Chemical®)) for 1 min. Slips were again washed twice in dH2O for 1 min and dried overnight at 37° C. Cells were grown to confluency on these cover slips and, following treatment, were decellularized. This was achieved by consecutive washes in Hank's Balanced Salt Solution (HBSS), 20 mM ammonium hydroxide (Sigma®) with 0.05% Triton X-100, and finally HBSS again. Matrices were fixed and stained as described above with chamber slides.

Western Blotting

Cells were treated with 10 ng/ml MMP-3 for 24 h in serum-free media. Media supernatants were aspirated and mixed 1:6 with StrataClean™ resin (Agilent®). After centrifugation, the supernatant was removed and the pellet was re-suspended in NP-40 lysis buffer containing 50 mM Tris pH 7.5, 150 mM NaCL, 1% NP-40, 10% SDS, 1× protease inhibitor (Roche®). Cells were lysed using NP-40 lysis buffer for protein collection. Samples were centrifuged at 10,000 rpm for 15 min (LabbIEC® Micromax microcentrifuge) and supernatant was retained. Protein samples were loaded onto a 10% SDS-PAGE gel at 30-50 μg per well. Proteins were separated by electrophoresis over the course of 150 min at constant voltage (120 V) under reducing conditions and subsequently electro-transferred onto methanol-activated PVDF membranes at constant voltage (12 V). Gels intended for use with Collagen IV antibodies were run under native conditions. Membranes were blocked for 1 h at room temperature in 5% non-fat dry milk and incubated overnight at 4° C. with rabbit primary antibodies to collagen IV, α-SMA, laminin and fibronectin as previously stated at concentrations of 1 in 1000 but 1 in 500 for laminin. Membrane blots were washed 3×5 min in TBS and incubated at room temperature for 2 h with horse radish peroxidase-conjugated anti-rabbit secondary antibody (Abcam®). Blots were again washed and treated with a chemiluminescent substrate (WesternBright ECL, Advansta®) and developed on a blot scanner (C-DiGit™,) LI-COR®). The membranes containing cell lysate samples were re-probed with GAPDH antibody (ab9485, Abcam®) for loading control normalization. Media samples were normalized against their total protein concentration as determined by a spectrophotometer (ND-1000™, NanoDrop®). A total of four replicate blots were quantified for each cell lysate sample antibody, and 2-3 replicates for a media sample. Band images were quantified using ImageJ software. Fold change in band intensity was represented in comparison to vehicle control treatments of PBS.

Adeno-Associated Virus (AAV)

AAV-2/9 containing the enhanced green fluorescent protein (eGFP) reporter gene (Vector Biolabs®) was initially used to assess viral transduction and expression in the anterior chambers of wild type mice (C57/BL6). Murine MMP-3 cDNA was incorporated into Bam HI/Xhol sites of the pAAV-MCS vector (Cell Biolabs Inc®) for constitutive expression of MMP-3. A null virus was used as contralateral control using the same capsid and vector. The inducible vector was designed by cloning MMP-3 cDNA into a pSingle-tTS (Clontech®) vector. This vector was then digested with BsrBI and BsrGI and the fragment containing the inducible system and MMP-3 cDNA was ligated into the NotI site of expression vector pAAV-MCS, to incorporate left and right AAV inverted terminal repeats (L-IRT and R-ITR). AAV-2/9 was generated using a triple transfection system in a stable HEK-293 cell line (Vector Biolabs®). For animals injected with the inducible virus, after a 3-week incubation period, 0.2% doxycycline (D9891, Sigma®) in PBS was administered twice daily to the eye for 10-16 days to induce viral expression. A similar inducible virus expressing eGFP was used as a control in the inducible study.

Intracameral Injection

Animals were anaesthetized by intra-peritoneal injection of ketamine (Vetalar V™, Zoetis®) and domitor (SedaStart™, Animalcare®) (66.6 and 0.66 mg/kg, respectively). Pupils were dilated using one drop of tropicamide and phenylephrine (Bausch & Lomb®) on each eye. 2 μl of virus at a stock titre of 5×10¹³ vector genomes per ml was initially back-filled into a glass needle (ID-1.0 mm, WPI) attached via tubing (ID-1.02 mm, OD-1.98 mm, Smiths) to a syringe pump (PHD Ultra™, Harvard Apparatus®). An additional 1 μl of air was then withdrawn into the needle. Animals were injected intracamerally just above the limbus. Viral solution was infused at a rate of 1.5 μl/min for a total of 3 μl to include the air bubble. Contralateral eyes received an equal volume and titre of either AAV-MMP-3 or AAV-Null. The air bubble prevented the reflux of virus/aqueous back through the injection site when the needle was removed. Fucidic gel (Fucithalmic Vet™, Dechra®) was applied topically following injection as an antibiotic agent. To counter anaesthetic, Antisedan (atipamezole hydrochloride, SedaStop™, Animalcare®) was intra-peritoneally injected (8.33 mg/kg) and a carbomer based moisturizing gel (Vidisic™, Bausch & Lomb®) was applied during recovery to prevent corneal dehydration.

Immunohistochemistry (Mouse Eyes)

Eyes were enucleated 4 weeks post-injection of virus and fixed in 4% paraformaldehyde overnight at 4° C. The posterior segment was removed by dissection and anterior segments were washed in PBS and placed in a sucrose gradient of incrementing sucrose concentrations containing 10%, 20% and finally 30% sucrose in PBS. Anterior segments were frozen in O.C.T compound (VWR Chemicals®) in an isopropanol bath immersed in liquid nitrogen and cryosectioned (CM 1900, Leica Microsystems®) at 12 μm thick sections. Sections were gathered onto charged Polysine® slides (Menzel-Glaser®) and blocked for 1 h with 5% normal goat serum (Ser. No. 10/658,654, Fischer Scientific®) and 0.1% Triton X-100 in PBS. Slides were incubated overnight at 4° C. in a humidity chamber with a 1:100 dilution of primary antibody. Antibodies used were MMP-3 (ab52915, Abcam®) and GFP (Cell Signalling®). Sections were washed three times in PBS for 5 min and incubated with a Cy-3 conjugated anti-rabbit IgG antibody (ab6936, Abcam®) at a 1:500 dilution for 2 h at 37° C. in a humidity chamber. Slides were washed as before and counter stained with DAPI for 30 s. Slides were mounted using Aquamount (Hs-106, National Diagnostics®) with coverslips (Deckglaser®) and visualized using a confocal microscope (Zeiss® LSM 710).

Total MMP-3 Quantification

MMP-3 concentration was quantified using enzyme-linked immunosorbent assay (ELISA) kits for both human SC monolayers (DMP300, R&D Systems®) and murine aqueous (RAB0368-1KT, Sigma®) according to the manufacturer's protocol. SC monolayers were cultured and treated with a 1 in 10 dilution of human cataract and POAG AH, a method previously described. Media was taken from the monolayers 24 h post-treatment and assayed for total MMP-3.

To measure the secretion of MMP-3 by AAV-2/9 into the AH, animals were inoculated with virus as described previously via intracameral injection. Four weeks post-injection, the animals were sacrificed and AH was collected. This was achieved by the cannulation of the cornea with a pulled glass needle (1B100-6, WPI®) and gentle pressing of the eye until it was deflated. Aqueous was expelled from the needle (approximately 5 μl) by the attachment of a 25 ml syringe connected via barb fitting and tubing (Smiths Medical®) and a gradual push of the syringe plunger. Aqueous was assayed using the previously mentioned ELISA kit.

MMP-3 Activity Assay (FRET)

Enzymatic activity of secreted MMP-3 was quantified using fluorescence resonance energy transfer (FRET). A fluorescent peptide consisting of a donor/acceptor pair remains quenched in its intact state. This peptide contains binding sites specific to MMP-3. Once cleavage occurs through MMP-3 mediated proteolysis, fluorescence is recovered by the transfer of energy from the donor to the acceptor, resulting in an increase in the acceptor's emission intensity. Cleavage of substrate, and therefore fluorescence, was monitored on a FLUOstar OPTIMA (BMG Labtech®) over the course of 2.5 h at 37° C., to allow ample time for substrate cleavage. Media samples were collected from treated SC monolayers and combined with a 1:100 dilution of an MMP-3 specific substrate (ab112148, Abcam®). Levels of active MMP-3 were interpolated from a standard curve defined by ELISA. For murine aqueous MMP-3 activity, aqueous was retrieved four weeks post-injection of AAV-MMP-3 or AAV-Null as described above. Aqueous samples were processed through an activity kit (abe3730, Source Bioscience®), selected for its high sensitivity and specificity, according to the manufacturer's protocol.

Enzymatic activity was calculated as described in MMP-3 activity Assay Kit's (ab118972, Abcam®) protocol:

$$MMP - 3 Activity ( nmol \text{/} min \text{/} ml ) = \frac{B \times Dilution Factor}{( T 2 - T 1 ) \times V} ,$$

Where B is the level of MMP-3 interpolated from the standard curve, T1 is the time (min) of the initial reading, T2 is the time (min) of the second reading and V is the sample volume (ml) added to the reaction well. The units ‘nmol/min/ml’ are equivalent to ‘mU/ml’.

Measurement of Outflow Facility

Animals were sacrificed for outflow facility measurement 4 weeks after injection of virus. Eyes were enucleated for ex vivo perfusion using the iPerfusion™ system. Contralateral eyes were perfused simultaneously using two independent but identical ierfusion systems. Each system comprises an automated pressure reservoir, a thermal flow sensor (SLG64-0075, Sensiron®) and a wet-wet differential pressure transducer (PX409, Omegadyne®), in order to apply a desired pressure, measure flow rate out of the system and measure the intraocular pressure respectively. Enucleated eyes were secured to a pedestal using a small amount of cyanoacrylate glue in a PBS bath regulated at 35° C. Perfusate was prepared (PBS including divalent cations and 5.5 mM glucose) and filtered (0.2 μm, GVS Filter Technology®) before use. Eyes were cannulated using a beveled needle (NF33BV NanoFil™, World Precision Instruments®) with the aid of a stereomicroscope and micromanipulator (World Precision Instrumente). Eyes were perfused for 30 min at a pressure of −8 mmHg in order to acclimatise to the environment. Incrementing pressure steps were applied from 4.5 to 21 mmHg, while recording flow rate and pressure. Flow (Q) and pressure (P) were averaged over 4 min of steady data, and a power law model of the form

$$Q = {{C}_{r} ( \frac{P}{{P}_{r}} )}^{β} P$$

was fit to the data using weighted power law regression, yielding values of Cr, the reference facility at reference pressure Pr=8 mmHg (corresponding to the physiological pressure drop across the outflow pathway), and #, a nonlinearity parameter characterizing the pressure-dependent increase in facility observed in mouse eyes.

Intraocular Pressure (IOP)

IOP measurements were performed by rebound tonometry (TonoLab™, Icare®) both prior to intracameral injection and 4 weeks post-injection. Readings, which were the average IOP values after five tonometric events, were taken 10 min after the intraperitoneal administration of mild general anaesthetic (53.28 mg/kg ketamine and 0.528 mg/kg domitor). Two readings were taken for one eye, then the other. This was repeated for a total of four readings per eye. Due to a minimum reading of 7 mmHg by the tonometer, a non-parametric approach was taken in the analysis of the readings. The median IOP was calculated for each eye, and MAD (median absolute deviation) values were used as a measure of dispersion. For comparing median values in a paired population, the Wilcoxon matched-pairs signed-rank test was employed to test for changes in IOP pre- and post-injection, and also for changes between contralateral eyes.

Analysis of Central Corneal Thickness

Enucleated mouse eyes transduced with AAV-MMP-3 or its contralateral control, AAV-Null, were fixed overnight in 4% PFA and washed in PBS. Posterior segments were removed by dissection under the microscope and anterior segments were embedded in medium (Tissue-Tek® OCT Compound™). Serial sectioning was performed on each eye and five frozen sections (12 μm) were transferred to a Polysine slide (Thermo Scientific®) for staining with DAPI and mounted with aqua-polymount (Polyscience®). Corneal sections were judged to be central by qualitatively taking the same distance from both iridocorneal angles. For quantitation, we measured the corneal thickness of sections on five consecutive slides by light and confocal microscopy (Zeiss® LSM 710). A total of 25 measurements were taken from each eye to represent mean central corneal thickness (μm) using the NIH ImageJ software.

Transmission Electron Microscopy

Ultrastructural investigation was performed by transmission electron microscopy (TEM) in four pairs of mouse eyes. One eye of each pair was injected with AAV-Null, the other with AAV-MMP-3, as described above. Four weeks after injection, the eyes were enucleated and immersion fixed in Karnovsky's fixative (2.5% PFA, 0.1 M cacodylate, 2.25% glutaraldehyde and dH₂O) for 1 h. Eyes were then removed from fixative and the cornea pierced using a 30-gauge needle (BD Microlance 3™, Becton Dickinson®). Eyes were placed back into fixative overnight at 4° C., washed 3×10 min, stored in 0.1 M cacodylate.

Here the eyes were cut meridionally through the center of the pupil, the lens carefully removed, and the two halves of each eye embedded in Epon. Semi-thin sagittal and then ultra-thin sections of Schlemm's Canal (SC) and trabecular meshwork (TM) were cut from one end of each half, and then the other approximately 0.2-0.3 mm deeper. The location of the superficial and deeper cut ends was alternated for the second half of the eye such that all four regions examined were at least 0.2-0.3 mm distant from one another. The ultrathin sections contained the entire anterior posterior length of the inner wall and the TM.

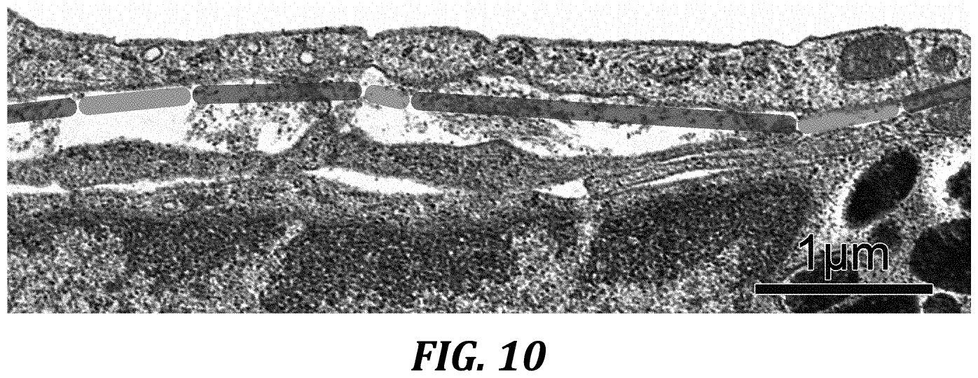

In four regions of each eye, we measured the length of optically empty space immediately underlying the inner wall endothelium of SC (FIG. 10). We also measured the inner wall length in contact with ECM, including basement membrane material, elastic fibres, or amorphous material. The optically empty length divided by the total length (optically empty+ECM lengths) was calculated and defined as the percentage of optically empty length for that region. All measurements were performed at 10,000× magnification, with each region including approximately 100 individual lengths of ECM or optically empty space.

Statistical Analysis

For TEER values, activity units (mU/ml) and concentrations (ng/ml), statistical differences were analyzed by using unpaired two-tailed Student's t-tests. Differences in Pₐₚₚ values (cm/s) were determined by a one way ANOVA with Tukey's correction for multiple comparisons, where appropriate. ELISA standard curve concentrations were log-transformed and absorbance values were fitted to a sigmoidal dose response curve with variable slope for interpolation. Fold change of western blot data was log-transformed and investigated for significance using a one-sample t-test against a theoretical mean of 0. To measure MMP-3 concentration and activity in the AH of wild type (WT) mice, a paired two-tailed t-test was carried out for contralateral samples. Outflow facility was analyzed using a weighted paired t-test performed in MATLAB, incorporating both system and biological uncertainties. For IOP data, median values were obtained to reflect the non-parametric nature of the tonometer, and the Wilcoxon matched-pairs signed rank test was used to compare changes in paired populations. For morphology, the distribution of values representing the % optically empty length was first examined using a Shapiro-Wilk and Anderson-Darling tests to detect for deviations from a normal distribution. The % optically empty length between contralateral eyes was then analyzed using a paired Student's t-test. Statistical significance was inferred when P<0.05 in all experimentation. Results were depicted as ‘mean, (95% Confidence Intervals)’ unless otherwise stated in the results section.

### Example 9: Inducible MMP-3 Expression in a Murine Model of Steroid-Induced Glaucoma

To assess the effect of the inducible MMP-3 virus on IOP and outflow facility in a mouse model of glaucoma, the glucocorticoid-induced ocular hypertension (OHT) model was used. This OHT model should reflect more accurately the degree to which MMP-3 might be effective in a glaucomatous environment.

### Methods

Intracameral Injection

Mice were intracamerally injected with AAV-iMMP-3 in one eye and AAV-iGFP in the contralateral eye as a control as follows. Mice were anaesthetized by isoflurane in a chamber for two minutes before being transferred to a headholder. Aqueous humor was withdrawn using a glass capillary needle and injected, through the same intracameral site, with approximately 4 μl of 1×10¹² viral genomes per ml, using a syringe held by a micromanipulator. This was left in the eye for a minute to acclimatize and a drop of fucithalmic (antibacterial) was placed on the eye before the needle was withdrawn.

Implantation

Two weeks after intracameral inoculation of virus, animals were again put under anaesthesia, subcutaneously injected with 100 μl the antibiotic Enrocare® Enrofloxacin and intramuscularly injected with 40 ul the painkiller Bupracare® Buprenorphine. Osmotic pumps were filled with reconstituted dexamethasone to account for a delivery of 2 mg/kg/day and inserted subcutaneously into the lower back. Mice were given Complan® meal replacement shake to avoid weight loss. Mice were treated with dexamethasone for a total of 4 weeks.

Intraocular Pressure (IOP)

A method was developed to best account for current limitations in IOP measurement. Such limitations include the effect of anaesthesia on IOP, IOP decay at the onset of anaesthesia, environmental stresses, a minimum value of 6 mmHg readable by the Icare® TONOLAB™ tonometer, and the inherent variation of tonometry itself. Temperature readings were monitored every day for a month leading up to, and for, the duration of the experiment to ensure no major fluctuations were observed. Animals were allowed to acclimatize for 3 weeks prior to experimentation. Animals were anaesthetized using 3% isoflurane in a chamber, and after 2 minutes were transferred to a head holder with inlets and outlets to the isofluorane vaporizer and scavenger. Tonometry measurements were taken every minute from minute 3 to minute 8, alternating between each eye every minute. Animals were measured in the OD eye first, but the first eye to be measured was alternated each week. Each tonometry measurement was the average of 5 individual readings, as determined by the Tonolab. A total of 3 measurements were taken at each minute timepoint. Values were imported to excel and all post-processing was performed through MATLAB® mathematical analysis software. A Shapiro-Wilks test was implemented initially to test for normality. As the distribution was non-normal, and to account for the non-parametric nature of the tonometer, central tendencies were determined by the median, and all statistical tests used were non-parametric tests. The median IOP for each timepoint was calculated for each eye in all animals, and interpolated to 5 minutes. Eyes treated with iMMP-3 and iGFP were statistically compared using a Wilcoxon matched pairs signed rank test on median IOP changes over the course of the 6 weeks, or on median IOP of the final week alone. A 1-sample Wilcoxon test was employed to test the significance of the median change in IOP over the timecourse versus a hypothetical median IOP change of 0 mmHg. Unpaired comparisons between dexamethasone groups were made using a Wilcoxon rank sum test.

Ocular Perfusion

A day was allowed for corneal recovery after the final IOP measurement, after which animals were sacrificed and eyes enucleated for ex vivo perfusion using the iPerfusion™ outflow measurement system. Eyes were mounted onto platforms in perfusion chambers regulated at 35 degrees and cannulated with a glass microneedle on a micromanipulator. Eyes were perfused at 8 mmHg for 30 minutes for acclimatization. Incrementing pressure steps were applied from 4.5 to 21 mmHg. Flow and pressure were averaged over the course of 4 minutes of steady data and a power law model fit to the data. Facility values were obtained from a reference pressure of 8 mmHg and analyzed using a weighted t-test, as described in Sherwood et al., “Measurement of Outflow Facility Using iPerfusion,” PLoS ONE 11(3):e0150694, doi:10.1371/journal.pone.0150694 (2016).

Electron Microscopy

Ultrastructural analysis is performed by transmission electron microscopy. Eyes were fixed in Karnovskys fixative overnight and then transferred into 0.1M cacodylate. Semi- and ultra-thin sections is cut and the length of optically empty space underlying the inner wall endothelium was measured.

### Results

MMP-3 Reduces IOP in a Steroid Model

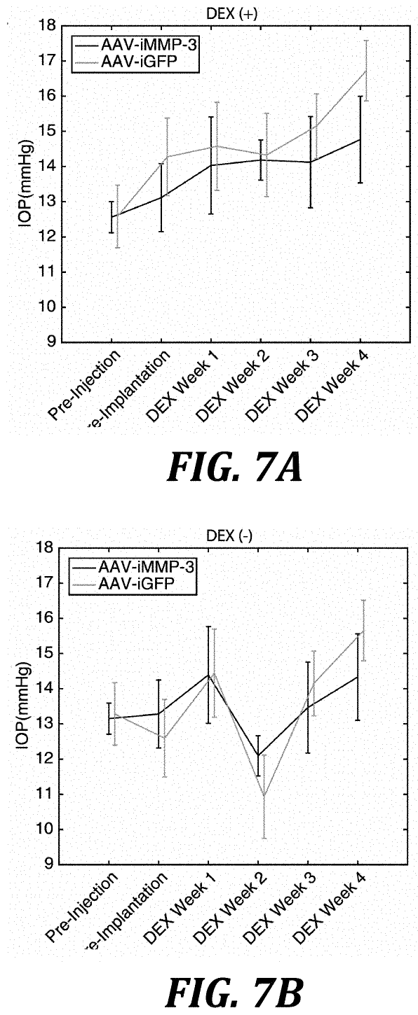

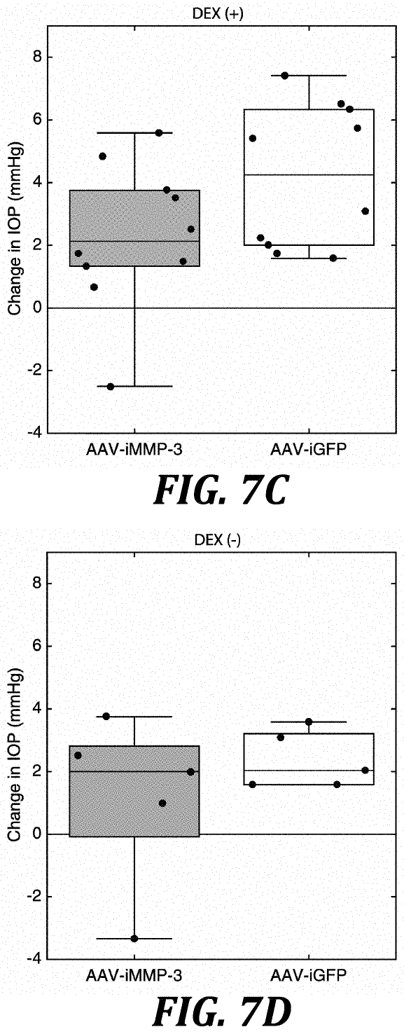

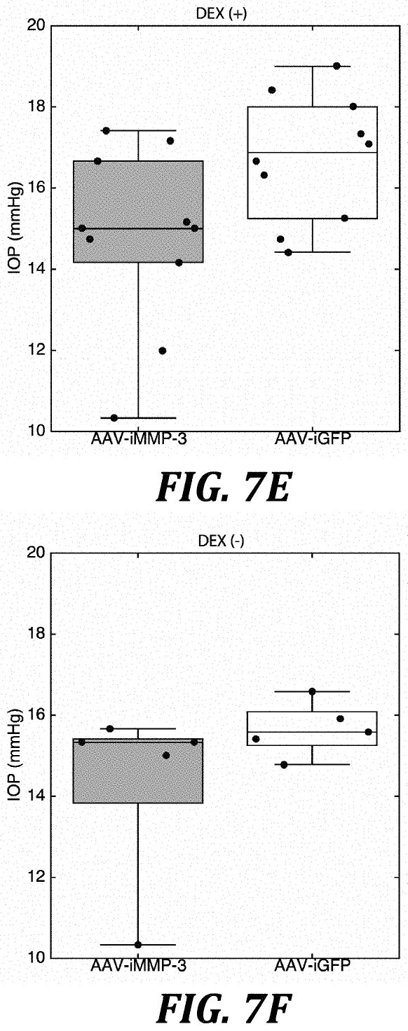

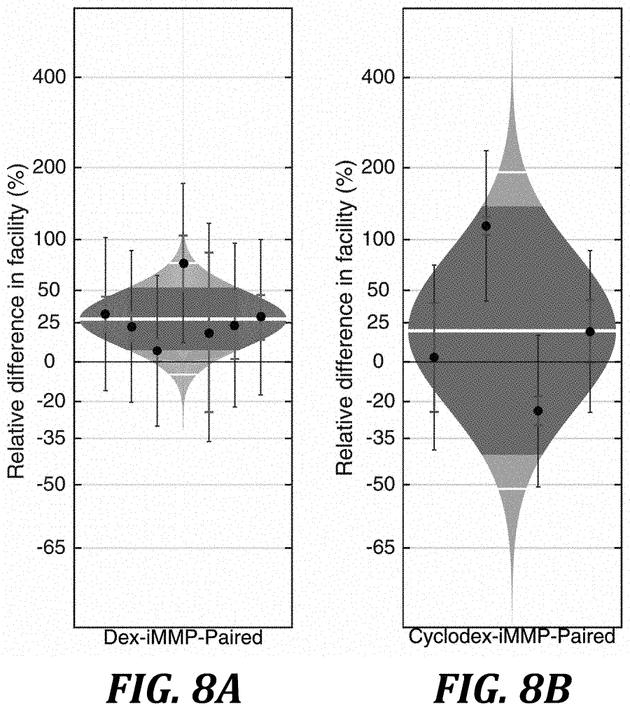

IOP measurements were taken each week for a total of 6 weeks until completion of the experiment. This was visualized as mean IOP and 95% CI of animals for each week (FIGS. 7A-7B). Changes in IOP were analyzed using medians to account for the non-parametric distribution of IOP data and the inability of the tonolab to read below 6 mmHg. Dexamethasone increased median IOP over time for a total change of 4.25 mmHg. This was compared to an assumed baseline of 0 change, and analyzed via a 1-sample Wilcoxon signed rank test with a hypothetical median of 0. Both eyes in the dexamethasone-treated animals were significantly increased over time; however, contralateral eyes were significantly different when tested with a Wilcoxon signed rank matched paired test. MMP-3 treated eyes in this group had a median change of 2.13 over time, representing a 2.12 mmHg difference compared to GFP-treated eyes (FIG. 7C). A difference in IOP was not observed in normotensive animals between contralateral eyes (FIG. 7D), although a change was observed in both eyes of 2 mmHg from the initial IOP measurement. Analysis of IOP measurements at the final timepoint alone show similar characteristics. In dex-treated animals (FIG. 7E), median IOP stands at 15 and 16.88 mmHg for both MMP-3 and GFP treated eyes respectively. This represents a significant difference at P=0.014, n=10. In cyclodextrin-treated animals (FIG. 7F), a median IOP of 15.33 (MMP-3) and 15.58 (GFP) is observed. This is a non-significant difference at P=0.188, n=5 using the Wilcoxon signed rank matched pairs test.

MMP-3 Increases Outflow Facility in a Steroid Model

In dex-induced animals, a 28 [8, 52] % difference was observed in outflow facility between iMMP-3 and iGFP treated eyes. This was significant at P=0.024, n=7. The cyclodextrin control group showed a similar difference in facility of 20 [−41, 142] %, but was not significant at P=0.476, n=4. The higher confidence interval range and lack of significance is likely due to the low n of this group.

While illustrative embodiments have been illustrated and described, it will be appreciated that various changes can be made therein without departing from the spirit and scope of the invention.

1. A recombinant AAV (rAAV) vector comprising a polynucleotide sequence encoding matrix metalloproteinase 3 (MMP-3).

2. The rAAV vector of claim 1, wherein said rAAV vector comprises a genome selected from the groups consisting of a single-stranded genome and a self-complementary genome.

3-5. (canceled)

6. The rAAV vector of claim 1, wherein the polynucleotide sequence encoding matrix metalloproteinase 3 (MMP-3) is operably linked to a CMV promoter.

7. The rAAV vector of claim 1, wherein the polynucleotide sequence encoding MMP-3 comprises a nucleotide sequence at least 95% identical to SEQ ID NO: 1.

8. (canceled)

9. The rAAV vector of claim 1, wherein the rAAV vector comprises a capsid selected from the group consisting of AAV1, AAV2, AAV4, AAV5, AAV6, AAV7, AAV8, AAV9, AAV10, AAV11, AAV12, AAV13, and Anc80L65.

10. The rAAV vector of claim 9, wherein the rAAV vector is of the serotype AAV9.

11-12. (canceled)

13. The rAAV vector of claim 10 comprising the nucleotide sequence set forth in SEQ ID NO: 1.

14. The rAAV vector of claim 1, wherein contacting the rAAV vector to a human trabecular meshwork (HTM) monolayer increases the rate of tracer molecule flux through said monolayer by more than about 10% over the tracer molecule flux through a HTM monolayer not contacted with said rAAV.

15. The rAAV vector of claim 1, wherein contacting said rAAV vector to a human travecular meshwork (HTM) monolayer decreases the transendothelial electrical resistance (TEER) of said monolayer by more than about 10 Ohm per cm², more than about 15 Ohm per cm², or more than about 20 Ohm per cm² over the TEER of a monolayer not contacted with said rAAV.

16. A method of treating a vision disorder in a subject suffering from the vision disorder, comprising administering to an eye of the subject a therapeutically effective amount of a recombinant AAV (rAAV) comprising a polynucleotide sequence encoding matrix metalloproteinase 3 (MMP-3).

17. The method of claim 16, wherein the polynucleotide sequence encoding MMP-3 comprises a nucleotide sequence at least 95% identical to SEQ ID NO: 1.

18. The method of claim 17, wherein the rAAV vector is of the serotype AAV9.

19-20. (canceled)

21. The method of claim 16, wherein administering the rAAV to said eye increases outflow of said eye.

22. The method of claim 16, wherein administering the rAAV to said eye decreases intraocular pressure (IOP) of said eye.

23-27. (canceled)

28. A method of treating a vision disorder in a mammal, comprising injecting a therapeutic composition comprising a rAAV vector into the anterior chamber of said mammal's eye,
wherein the rAAV vector transduces cells in the anterior chamber;
wherein the transduced cells secrete a therapeutic protein;
wherein the therapeutic protein modifies the extracellular matrix of the trabecular meshwork of said mammal's eye;
wherein said method treats said vision disorder in said mammal.

29. (canceled)

30. The method of claim 28, wherein said therapeutic protein is a matrix metalloproteinase (MMP).

31. The method of claim 30, wherein said MMP is MMP-3.

32-36. (canceled)

37. The method of claim 28, wherein the MMP-3 concentration in aqueous humor of said eye is increased by about 0.49 ng/ml or greater.

38-41. (canceled)

42. The method of claim 16, wherein the vision disorder is glaucoma.
